(19) United States
(12) Patent Application Publication
(10) Pub. No.: US 2008/0194024 A1
(43) Pub. Date: Aug. 14, 2008

(54) Culture of Non-Embryonic Cells at High Cell Density

(76) Inventor: Robert W. Mays, Shaker Heights, OH (US)

Correspondence Address: Anne Brown;Thompson Hine LLP, P.O. Box 8801, Dayton, OH, 45401-8801, US

(21) Appl. No.: 11/996,882

(22) PCT Filed: Jul. 31, 2006
(86) PCT No.: PCT/US06/29547

§ 371 Date: Jan. 25, 2008

Related U.S. Application Data
(60) Provisional application No. 60/703,823, filed on Jul. 29, 2005.

(51) Int. Cl. C12N 5/00

(52) U.S. Cl. 435/377

(57) ABSTRACT

The present invention is directed to the culture of non-embryonic cells, that can differentiate into cell types of more than one embryonic lineage, at high densities in culture under conditions that maintain differentiation capacity during expansion; more particularly, culturing non-embryonic cells at high densities in the presence of a GKS-3 inhibitor, such as BIO.

### RELATED APPLICATION

This application claims priority from U.S. Provisional Application Ser. No. 60/703,823 filed Jul. 29, 2005, which application is herein incorporated by reference.

### STATEMENT OF GOVERNMENT RIGHTS

This work was funded by United States Grant No. R01 DK58295 (NIH). The government may have certain rights to this invention.

### FIELD OF THE INVENTION

The present invention relates to the growth of cells in culture, specifically to the culture of non-embryonic cells that can differentiate into more than one embryonic lineage, at high density in the presence of at least one GSK3 inhibitor, such as 6-bromoindirubin-3′-oxime (also known as BIO).

### BACKGROUND OF THE INVENTION

Limitations in the development of therapies based on embryonic and adult stem cells include the inability to grow the cells at high density and the associated costs of large-scale cell culture. Coupled with the necessity for supplementing stem cell culture medias with exogenous cytokines and growth factors to maintain the cells in a pluripotent state, this makes the routine maintenance, study and use of stem cells time, space and cost prohibitive. Three of the four key scientific questions identified as “Barriers to Progress in Stem Cell Research for Regenerative Medicine” by the 2002 Committee on the Biological and Biomedical Applications of Stem Cell Research are applicable to ex vivo culturing issues: what causes stem cells to maintain an undifferentiated state, what cues do stem cells use to start and stop dividing, and what signals affect/initiate differentiation. Answering or gaining insight into any or all of these questions will aid in the realization of the therapeutic potential of stem cells and stem cell derived products, as well as define potential commercial products in the form of, for example, small molecule modulators.

The screening of small molecule libraries in high throughput drug discovery campaigns is the overriding paradigm for identifying and developing new therapeutics in the pharmaceutical industry. Once enough data has accumulated demonstrating a protein or pathway is implicated and validated in the biology of a disease, the target is assayed versus tens of thousands of compounds in an effort to find specific small molecule modulators of the target. Depending on the biology, either agonists or antagonists may be required to modulate the target and the pathway of interest in attempts to generate a novel therapeutic compound.

The idea of screening stem cells in a high throughput format to identify small molecule regulators of pluripotency is fairly novel, and until recently has been hampered by the scarcity of readily available stem cell sources as well as basic scientific hurdles (Horrocks, C., et al. (2003); McNeish, J. (2004)). Most research in the stem cell field has focused on identifying and isolating stem cells, in and of themselves. Historically, research has then progressed to determining the pluripotency or differentiated “nature” of a particular stem cell, the conditions required for maintaining its most undifferentiated state, the markers delineating stem cells from differentiated siblings, and then finally identifying the targets and pathways regulating pluripotency. Only recently has enough progress been made into understanding pathways mediating stem cell pluripotency to even contemplate a search for small molecule regulators.

However, within the last few years, the first reports of successful screening of small molecule libraries against stem cells to identify effectors of differentiation have been published. Screening of a mouse embryonic mesoderm stem cell line against a library of 50,000 compounds identified a novel small molecule agonist that induced differentiation of the cells into an osteogeneic lineage (Wu, X., et al. (2002)). When tested against several other mouse mesenchymal cell lines, the compound induced the formation of pre-adipocytes and myoblasts, indicating that it may bind to and activate multiple receptors and pathways in different cells or that the same differentiation pathway may induce multiple endpoints in different cell lines. This group has also screened a second small molecule library of 100,000 compounds and identified another chemical entity that acts to induce cardiomyogenesis in another stem cell line (Wu, X., et al. (2004)).

Recent reports indicate that using stem cells to screen for small molecule drugs that act to rescue or maintain pluripotent cell phenotypes are also possible and can be successful. The screening of a focused small molecule drug library against a mouse mesodermal stem cell line, which had already been differentiated into a myoblastic lineage, led to the identification of the small molecule “reversine” (Chen, S., et al. (2003)). This molecule acted to “de-differentiate” the cell line from a committed myoblast, back to the multipotent precursor, which could then be successfully induced to differentiate into 3 different lineages.

One interesting result centers on the identification of BIO as a small molecule regulator of pluripotency in both mouse and human embryonic stem cell lines. Following the identification of the Wnt family of proteins, there has been a great deal of focus on understanding the role of Wnt signaling in cell biological processes. Wnts are expressed in a diverse set of tissues and influence numerous processes in development, including segment polarity in Drosophila and limb and axis development in vertebrates (Cadigan and Nusse (1997)). Dysregulation of the Wnt signaling pathway plays an oncogenic role in colon, breast, prostate and skin cancers (Polakis (2000)). More recently the canonical Wnt signaling pathway has been identified as having a role in the maintenance of pluripotency in a variety of stem cells (Zhu and Watt (1999); Korinek et al. (1998); Chenn and Walsh (2002)).

Biologically, Wnts act by binding to two types of receptor molecules at the cell surface. One is the Frizzled (Fz) family of seven-pass transmembrane proteins (Wodarz and Nusse (1998)), the second a subset of the low-density lipoprotein receptor related protein (LRP) family (Pinson et al. (2000)). Experiments have demonstrated that both Fz and LRP are needed to activate the downstream components of the canonical pathway. In the absence of Wnt signaling, β-catenin is associated with a large multi-protein complex composed of adenomatous polyposis coli (APC), axin and glycogen synthase kinase 3β (GSK-3β. In this complex, β-catenin is phosphorylated at its amino terminus by GSK-3β, targeting it for ubiquitination and degradation by proteosomes (Cadigan and Nusse (1997)).

Binding of Wnt to the co-receptors results in recruitment of the protein Dsh (Disheveled), which relays the activation signal to the multi-protein complex. Dsh interacts with axin, thereby inhibiting GSK-3β from phosphorylating β-catenin and preventing its degradation (Willert and Nusse (1998)). This stabilization and accumulation of β-catenin results in its translocation to the nucleus, where it binds to members of the lymphoid enhancer factor/T-cell factor (LEF/TCF) family of transcription factors, subsequently inducing expression of their associated target genes (Eastman and Grosschedl, (1999)). Interestingly, two genes up regulated by Wnt through this pathway are Notch1 and HoxB4, genes previously implicated in the self-renewal of HSC (Reya et al. (2003)). Wnt signaling has also been implicated in the self-renewal of epidermal progenitor cells (Zhu and Watt (1999)), gastric stem cells (Korinek et al. 1998) and neural stem cells (Chenn and Walsh (2002)).

Recognizing GSK-3β plays a role in the canonical Wnt signaling pathway and therefore a potential target in cancer therapies, a group of biologists and chemists teamed up to screen a panel of naturally occurring small molecules to look for inhibitors of GSK-3β. A class of molecules called indirubins derived from Mediterranean mollusks was identified as having GSK-3β inhibitory activity. Synthesis of a defined library of synthetic indirubin analogues followed, with one molecule, BIO, having 100× specificity for GSK-3β over other related kinases, and an IC₅₀ in the nanomolar range (Meijer, L., et al. (2003)). Addition of BIO to developing Xenopus embryos indicated that BIO's activity mimicked Wnt signaling in developmental assays.

Subsequently, BIO was tested in both mouse and human embryonic stem (ES) cell culture systems to determine if it had an effect in mammalian embryonic systems, as well as to address the involvement of the Wnt signaling pathways in ES cells. BIO was able to substitute for the addition of feeder cultures or addition of exogenous cytokines in maintaining the ES cultures in an undifferentiated pluripotent state as demonstrated by the expression of the pluripotent state-specific transcription factors Oct-3a, Rex-1 and Nanog. In addition, BIO-mediated Wnt activation was functionally reversible, as withdrawal of the compound leads to normal multi-differentiation programs in both human and mouse embryonic stem cells.

### Stem Cells

The embryonic stem (ES) cell has unlimited self-renewal and can differentiate into all tissue types. ES cells are derived from the inner cell mass of the blastocyst or primordial germ cells from a post-implantation embryo (embryonic germ cells or EG cells). ES (and EG) cells can be identified by positive staining with antibodies to SSEA 1 (mouse) and SSEA 4 (human). At the molecular level, ES and EG cells express a number of transcription factors specific for these undifferentiated cells. These include Oct-4 and rex-1. Rex expression depends on Oct-4. Also found are the LIF-R (in mouse) and the transcription factors sox-2 and rox-1. Rox-1 and sox-2 are also expressed in non-ES cells. Another hallmark of ES cells is the presence of telomerase, which provides these cells with an unlimited self-renewal potential in vitro.

Oct-4 (Oct-3 in humans) is a transcription factor expressed in the pregastrulation embryo, early cleavage stage embryo, cells of the inner cell mass of the blastocyst, and embryonic carcinoma (EC) cells (Nichols J., et al. (1998)), and is down-regulated when cells are induced to differentiate. Expression of Oct-4 plays a role in determining early steps in embryogenesis and differentiation. Oct-4, in combination with Rox-1, causes transcriptional activation of the Zn-finger protein Rex-1, also required for maintaining ES in an undifferentiated state (Rosfjord and Rizzino A. (1997); Ben-Shushan E, et al. (1998)). In addition, sox-2, expressed in ES/EC, but also in other more differentiated cells, is needed together with Oct-4 to retain the undifferentiated state of ES/EC (Uwanogho D et al. (1995)). Maintenance of murine ES cells and primordial germ cells requires LIF.

The Oct-4 gene (Oct-3 in humans) is transcribed into at least two splice variants in humans, Oct-3A and Oct-3B. The Oct-3B splice variant is found in many differentiated cells whereas the Oct-3A splice variant (also designated Oct-3/4) is reported to be specific for the undifferentiated embryonic stem cell (Shimozaki et al. (2003)).

Adult stem cells have been identified in many tissues. Hematopoietic stem cells are mesoderm-derived and have been purified based on cell surface markers and functional characteristics. The hematopoietic stem cell, isolated from bone marrow, blood, cord blood, fetal liver and yolk sac, is the progenitor cell that reinitiates hematopoiesis and generates multiple hematopoietic lineages. Hematopoietic stem cells can repopulate the erythroid, neutrophil-macrophage, megakaryocyte and lymphoid hematopoietic cell pool.

Neural stem cells were initially identified in the subventricular zone and the olfactory bulb of fetal brain. Studies in rodents, non-human primates and humans, have shown that stem cells continue to be present in adult brain. These stem cells can proliferate in vivo and continuously regenerate at least some neuronal cells in vivo. When cultured ex vivo, neural stem cells can be induced to proliferate and differentiate into different types of neurons and glial cells. When transplanted into the brain, neural stem cells can engraft and generate neural cells and glial cells.

Mesenchymal stem cells (MSC), originally derived from the embryonal mesoderm and isolated from adult bone marrow, can differentiate to form muscle, bone, cartilage, fat, marrow stroma and tendon. Mesoderm also differentiates to visceral mesoderm, which can give rise to cardiac muscle, smooth muscle, or blood islands consisting of endothelium and hematopoietic progenitor cells. All of the many mesenchymal stem cells that have been described have demonstrated limited differentiation to cells generally considered to be of mesenchymal origin. To date, the best characterized mesenchymal stem cell reported is the cell isolated by Pittenger, et al. (1999) and U.S. Pat. No. 5,827,740 (CD105⁺ and CD73⁺). This cell is apparently limited in differentiation potential to cells of the mesenchymal lineage.

### SUMMARY OF THE INVENTION

One embodiment provides culture methods comprising culturing non-embryonic cells at a high cell density in the presence of at least one GSK-3 inhibitor, wherein said cells can differentiate into cell types of more than one embryonic lineage.

One embodiment provides a culture method comprising culturing non-embryonic cells at a density of at least about 8,000 cells/cm² in the presence of at least one GSK-3 inhibitor, wherein said cells can differentiate into cell types of more than one embryonic lineage. In one embodiment, the density of the cells is about 8,000 to at least about 50,000 cells/cm².

One embodiment provides a culture method comprising culturing non-embryonic cells at a density of at least about 50,000 cells/ml in the presence of at least one GSK-3 inhibitor, wherein said cells can differentiate into cell types of more than one embryonic lineage.

Another embodiment provides a culture method comprising culturing non-embryonic cells at a density of at least about 8,000 cells/cm² in the presence of at least one GSK-3 inhibitor, so that said cells maintain or increase their capacity to differentiate (potency) to a greater extent than said cells cultured in the absence of a GSK-3 inhibitor, where said cells can differentiate into cell types of more than one embryonic lineage.

Another embodiment provides a culture method comprising culturing non-embryonic cells at a density of least about 50,000 cells/ml in the presence of at least one GSK-3 inhibitor, so that said cells maintain or increase their capacity to differentiate (potency) to a greater extent than said cells cultured in the absence of a GSK-3 inhibitor, where said cells can differentiate into cell types of more than one embryonic lineage.

In one embodiment, the GSK-3 inhibitor is a compound of formula (I):

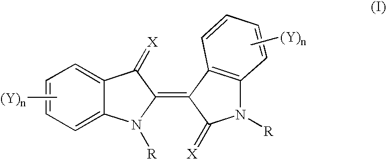

wherein each X is independently O, S, N—OR¹, N(Z), or two groups independently selected from H, F, Cl, Br, I, NO₂, phenyl, and (C₁-C₆)alkyl, wherein R¹ is hydrogen, (C₁-C₆)alkyl, or (C₁-C₆)alkyl-C(O)—;

each Y is independently H, (C₁-C₆)alkyl, (C₁-C₆)alkyl-C(O)—, (C₁-C₆)alkyl-C(O)O—, phenyl, N(Z)(Z), sulfonyl, phosphonyl, F, Cl, Br, or I;

each Z is independently H, (C₁-C₆)alkyl, phenyl, benzyl, or both Z groups together with the nitrogen to which they are attached form 5, 6, or 7-membered heterocycloalkyl;

each n is independently 0, 1, 2, 3, or 4;

each R is independently H, (C₁-C₆)alkyl, (C₁-C₆)alkyl-C(O)—, phenyl, benzyl, or benzoyl; and

wherein alkyl is branched or straight-chain, optionally substituted with 1, 2, 3, 4, or 50H, N(Z)(Z), (C₁-C₆)alkyl, phenyl, benzyl, F, Cl, Br, or I; and

wherein any phenyl, benzyl, or benzoyl is optionally substituted with 1, 2, 3, 4, or 50H, N(Z)(Z), (C₁-C₆)alkyl, F, Cl, Br, or I;

or a salt thereof.

In one embodiment, one X is O and the other X is N—OH. In another embodiment, one Y is Br. In another embodiment, one Y is Br at the 6′-position.

In one embodiment one n is 0 and the other n is 1. In another embodiment, each R is H.

In one embodiment, the GSK-3 inhibitor comprises:

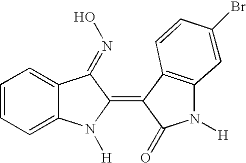

or a salt thereof. In one embodiment, the GSK-3 inhibitor comprises 6-bromoindirubin or 6-bromoindirubin-3′-oxime (BIO). In another embodiment, the GSK-3 inhibitor comprises LiCl, hymenialdisine, flavopiridol, kenpaullone, alsterpaullone, azakenpaullone, Indirubin-3′-oxime, 6-Bromoindirubin-3′-oxime (BIO), 6-Bromoindirubin-3′-acetoxime, Aloisine A, Aloisine B, TDZD8, compound 12, compound 1, Pyrazolopyridine 18, Pyrazolopyridine 9, Pyrazolopyridine 34, CHIR98014, CHIR99021, CHIR-637, CT20026, SU9516, ARA014418, Staurosporine, compound 5a, compound 29, compound 46, compound 8b, compound 17, compound 1A, GF109203x (bisindolyl-maleimide I), Ro318220 (bisindolyl-maleimide IX), SB216763, SB415286, CGP60474, TWS119, or a thiazolo 5,4-f quinazolin-9-one.

In one embodiment, the GSK-3 inhibitor is in the culture at a concentration of about 0.1 μM to about 1 μM. In another embodiment, the GSK-3 inhibitor is in the culture at a concentration of about 1 μM to about 2 μM. One embodiment provides removing or inactivating the GSK-3 inhibitor and culturing said cells to allow differentiation.

One embodiment provides a composition comprising non-embryonic cells at a density of at least about 8,000 cells/cm² in combination with at least one GSK-3 inhibitor, wherein said cells can differentiate into cell types of more than one embryonic lineage. In one embodiment, the density of the cells is about 8,000 cells/cm² to at least about 50,000 cells/cm². One embodiment provides a composition comprising non-embryonic cells at a density of at least about 50,000 cells/ml in combination with at least one GSK-3 inhibitor, wherein said cells can differentiate into cell types of more than one embryonic lineage. In one embodiment, the composition further comprises a carrier. In one embodiment, the carrier is cell culture medium. In another embodiment the carrier is a pharmaceutically acceptable carrier.

One embodiment provides a method to prepare a composition comprising admixing non-embryonic cells at a density of at least about 8,000 cells/cm² and at least one GSK-3 inhibitor, wherein said cells can differentiate into cell types of more than one embryonic lineage. In one embodiment, the density of the cells is about 8,000 cells/cm² to at least about 50,000 cells/cm². Another embodiment provides a method to prepare a composition comprising admixing non-embryonic cells at a density of at least about 50,000 cells/ml in combination with at least one GSK-3 inhibitor, wherein said cells can differentiate into cell types of more than one embryonic lineage. In one embodiment, the method further comprises admixing a carrier. In one embodiment, the carrier is a pharmaceutically acceptable carrier or cell culture medium.

The methods and compositions of the invention are applicable to all non-embryonic cells that can differentiate into cell types of more than one embryonic lineage. In one embodiment, the cell is a non-embryonic, non germ, non-embryonic germ cell that can form cell types of two or more embryonic lineages. Such a cell includes one that could form cell types of all three embryonic lineages, i.e., endoderm, ectoderm and mesoderm. The cell may express one or more of the genes reported to characterize the embryonic stem cell, i.e., telomerase or Oct-3A.

Cells for use in embodiments of the invention can be derived from any non-embryonic source, including any organ or tissue of a mammal, such as umbilical cord, umbilical cord blood, muscle, umbilical cord matrix, neural, placenta, bone, brain, kidney, liver, bone marrow, adipose, pancreas, oogonia, spermatogonia, or peripheral blood. In one embodiment, the mammal is a human, mouse, rat or swine.

One embodiment comprises transforming the cells with an expression vector comprising a preselected DNA sequence.

Another embodiment comprises culturing the cells in the presence of a cytokine or a growth factor. Another embodiment comprises differentiating the cells by contacting the cells with at least one differentiation factor.

### BRIEF DESCRIPTION OF THE DRAWINGS

FIG. 1 depicts a schematic of the experimental procedure.

FIG. 2 is a graphical representation of PCR data for gene expression in MAPCs treated with different concentrations of BIO.

FIG. 3 is a graphical representation of PCR data for Rex-1 gene expression in MAPCs treated with different concentrations of BIO.

FIG. 4 is a graphical representation of PCR data for AFP gene expression in MAPCs treated with different concentrations of BIO.

FIG. 5 is a graphical representation of PCR data for Sox-1 gene expression in MAPCs treated with different concentrations of BIO.

FIG. 6 depicts the FACS phenotype of mMAPCs treated with BIO.

FIG. 7 depicts (A) the morphology of mMAPCs treated with BIO and (B) the frequency of clusters after treatment with BIO.

FIG. 8 depicts the morphology of expanded high-density cultured mMAPCs treated with or without BIO (A). (B) Western blot of β-catenin protein expression in low-density cultured mMAPCs.

FIG. 9 depicts E-cadherin and phosphorylated β-catenin immunostaining of low density-cultured mMAPCs.

FIG. 10 depicts E-cadherin and phosphorylated β-catenin immunostaining of high-density-cultured mMAPCs.

FIG. 11 depicts the structure of some inhibitors of GSK-3 (Meijer, L. et al. (2004)).

### DETAILED DESCRIPTION OF THE INVENTION

One embodiment of the invention is directed to culture conditions for culturing non-embryonic cells, that can differentiate into cell types of more than one embryonic lineage, at high densities.

### Definitions

As used herein, the terms below are defined by the following meanings:

“MAPC” is an acronym for “multipotent adult progenitor cell.” It is used herein to refer to a non-embryonic stem (non-ES), non-germ, non-embryonic germ (non-EG) cell that can give rise to (differentiate into) cell types of more than one embryonic lineage. It can form cell lineages of at least two germ layers (i.e., endoderm, mesoderm and ectoderm) upon differentiation. Like embryonic stem cells, MAPCs from humans were reported to express telomerase or Oct-3/4 (i.e., Oct-3A). (Jiang, Y. et al. (2002)). Telomerase or Oct-3/4 have been recognized as genes that are primary products for the undifferentiated state. Telomerase is needed for self-renewal without replicative senescence. MAPCs derived from human, mouse, rat or other mammals appear to be the only normal, non-malignant, somatic cell (i.e., non-germ cell) known to date to express telomerase even in late passage cells. The telomeres are not sequentially reduced in length in MAPCs. MAPCs are karyotypically normal. MAPCs may express SSEA-4 and nanog. The term “adult,” with respect to MAPC is non-restrictive. It refers to a non-embryonic somatic cell.

“Multipotent” refers to the ability to give rise to cell types of more than one embryonic lineage. “Multipotent,” with respect to MAPC, is non-restrictive. MAPCs can form cell lineages of all three primitive germ layers (i.e., endoderm, mesoderm and ectoderm). The term “progenitor” as used in the acronym “MAPC” does not limit these cells to a particular lineage.

“Potency” refers to the differentiation capacity of a cell (e.g., the potential to differentiate into different cell types, for example, a multipotent cell can differentiate into cells derived from three germ layers). Potency can be demonstrated by testing for the expression of mRNAs and proteins associated with a pluripotent state, such as telomerase (TERT; telomerase is composed of two subunits, Telomerase Reverse Transcriptase (hTERT, the “h” is for human) and hTR (Telomerase RNA)) or Oct-3A. Another way is by testing for the presence/absence of markers (protein or mRNA) associated with a differentiated state (e.g., wherein the cell is committed to one embryonic lineage). The marker profile of cells can be determined by, for example, Q-PCR (e.g., of transcription factors), immunofluorescence, FACS analysis, Western blot or a combination thereof. Morphological assays can also be used to determine potency. These and other in vitro assays are known in the art (see, for example, WO 01/11011, which is incorporated herein by reference).

Additionally, cells can be assayed in vitro and in vivo to determine the cell's ability to differentiate (e.g., in response to stimuli (including, but not limited to, differentiation factors, growth factors, cytokines, culture conditions, or location in subject)). For example, potency can be demonstrated by exposing the cells to factors or cell culture conditions to differentiate the cells and then tested to determine if the cells have differentiated and to what cell type(s) they have differentiated (see, for example, WO 01/11011, which is incorporated herein by reference). Additionally, potency can be determined in vivo. For example, the cells can be placed (e.g., injected) in a subject (e.g., a NOD/SCID mouse). The cells can then be examined to determine if they differentiated and to what cell type(s) they have differentiated. The cells can be injected into a blastocyst. The cells of the developing or developed subject (e.g., mouse) can then be examined to determine if they differentiated and to what cell type(s) they have differentiated. For example, cells grown at various densities in the presence of a GSK-3 inhibitor can be tested for potency by injecting a cell (e.g., a genetically marked cell) into a mouse blastocyst, implanting the blastocyst, developing it to term and determining if the animal exhibits chimerism and in what tissues and organs the progeny are present (Jiang, Y. et al. (2002)). In vivo assays to determine potency are known in the art (see, for example, WO 01/11011).

A confluent state refers to a state when cells in culture come into contact with other cells in the same culture to form what appears to be a sheet of cells with few gaps between them (for adherent cells, such as a confluent monolayer (100% confluency)). Cells can form clusters prior to and at 100% confluency. Confluent cells are generally believed to no longer be in a growth phase. This observation is often related to the color of the media supporting the cells (i.e. rate of consumption) and the number of dead cells. The upper range of culturing cells in the methods of the invention is understood to be confluence (e.g., 100% confluency).

“Expansion” refers to the propagation of cells without differentiation.

“Self-renewal” refers to the ability to produce replicate daughter stem cells having differentiation potential that is identical to those from which they arose. A similar term used in this context is “proliferation.”

The term “isolated” refers to a cell or cells which are not associated with one or more cells or one or more cellular components that are associated with the cell or cells in vivo. An “enriched population” means a relative increase in numbers of the cell of interest, such as MAPCs, relative to one or more other cell types, such as non-MAPC cells types, in vivo or in primary culture.

“Differentiation factors” refer to cellular factors, such as growth factors (e.g., a substance which controls growth, division and maturation of cells and tissues, including, but not limited to, granulocyte-colony stimulating factor (G-CSF), granulocyte-macrophage colony stimulating factor (GM-CSF), nerve growth factor (NGF), neurotrophins, platelet-derived growth factor (PDGF), erythropoietin (EPO), thrombopoietin (TPO), myostatin (GDF-8), Growth Differentiation factor-9 (GDF9), basic fibroblast growth factor (bFGF or FGF2)) or angiogenic factors, which induce lineage commitment.

“Cytokines” refer to cellular factors that induce or enhance cellular movement, such as homing of MAPCs or other stem cells, progenitor cells or differentiated cells. Cytokines also include small proteins released by cells that have a specific effect on the interactions between cells, on communications between cells or on the behavior of cells. Cytokines include, but are not limited to, the interleukins, lymphokines and cell signal molecules, such as tumor necrosis factor and the interferons. Cytokines may also stimulate such cells to divide.

A “subject” or cell source can be vertebrate, preferably a mammal, more preferably a human. Mammals include, but are not limited to, humans, farm animals, sport animals and companion animals. Included in the term “animal” is dog, cat, fish, gerbil, guinea pig, hamster, horse, rabbit, swine, mouse, hamster, monkey (e.g., ape, gorilla, chimpanzee, orangutan), rat, sheep, goat, cow and bird.

An “effective amount” generally means an amount which provides the desired effect. For example, an effective amount is an amount of the desired compound sufficient to maintain or enhance the potency (differentiation capacity) of the cells in culture.

The terms “comprises”, “comprising”, and the like can have the meaning ascribed to them in U.S. Patent Law and can mean “includes”, “including” and the like. As used herein, “including” or “includes” or the like means including, without limitation.

### The Role of the Wnt Signaling Pathway in Maintaining the Pluripotency of Stem Cells

I) Introduction

Stem cells, regardless of the species, tissue of origin or stage of development at isolation, can be defined as cells that choose either self-renewal or differentiation as a means to renew another more specialized cell type. Although identified over 40 years ago, stem cells were, at the time, hard to reproducibly isolate and therefore minimally characterized and poorly understood. The identification and successful in vitro culturing of mouse ES (embryonic stem) cells, human ES cells, and most recently MAPC (Multipotent Adult Progenitor Cells), has given scientists a variety of homogeneous pluripotent cell types as tools to study the regulation of self-renewal versus differentiation. Subsequently, the last five years has seen a rapid advancement in the identification of proteins and signaling pathways involved in the biology of pluripotency.

II) The Role of the Wnt/β-catenin Signaling Pathway in Maintaining Pluripotency

Self-renewal versus differentiation decisions made by stem cells are the result of the intracellular processing of multiple independent extra-cellular cues working through defined signaling pathways. Although multiple other pathways, including the TGF-β and Stat 3 pathways, also play a role in regulating cell fate, the Wnt/β-catenin appears to have a major influence on the pluripotency of stem cells.

Wnt proteins represent a growing family of secreted signaling molecules expressed in a diverse set of tissues and have been shown to influence multiple processes in vertebrate and invertebrate development (Cadigan and Nusse 1997)). Aberrant Wnt signaling or dysregulation has been shown to contribute to a number of human cancers (Polakis (2000)). Recent in vivo and in vitro studies suggest that the canonical Wnt/β-catenin signaling pathway is also involved in regulating the self-renewal in stem cells (Sato et al. (2004)). Wnts act by binding to two types of receptor molecules at the cell surface. One is the Frizzled (Fz) family of seven-pass transmembrane proteins (Wodarz and Nusse (1998)), the second a subset of the low-density lipoprotein receptor related protein (LRP) family (Pinson et al. (2000)). Experiments have demonstrated that both Fz and LRP are needed to activate the downstream components of the canonical pathway. In the absence of Wnt signaling, β-catenin is associated with a large multi-protein complex composed of adenomatous polyposis coli (APC), axin and glycogen synthase kinase 3β (GSK3β). In this complex, β-catenin is phosphorylated at its amino terminus by GSK3β, targeting it for ubiquitination and degradation by proteosomes (Cadigan and Nusse (1997)).

Binding of Wnt to the co-receptors results in recruitment of the protein Dsh (Disheveled), and relay of the activation signal to the multi-protein complex. Dsh interacts with axin, thereby inhibiting GSK3β from phosphorylating β-catenin and preventing its degradation (Willert and Nusse (1998)). This stabilization and accumulation of β-catenin results in its translocation to the nucleus, where it binds to members of the lymphoid enhancer factor/T-cell factor (LEF/TCF) family of transcription factors, subsequently inducing expression of their associated target genes (Eastman and Grosschedl (1999)).

Interestingly, two genes up regulated by Wnt through this pathway are Notch1 and HoxB4, genes previously implicated in the self-renewal of HSC (Reya et al. (2003)). Wnt signaling has also been implicated in the self-renewal of epidermal progenitor cells (Zhu and Watt (1999)), gastric stem cells (Korinek et al. (1998)) and neural stem cells (Chenn and Walsh (2002)). Activation of the canonical Wnt pathway by inhibiting GSK3p activity was shown to be sufficient for maintaining pluripotency in both human and mouse ES cells in the absence of any other exogenous growth factors (Sato et al. (2004)). Most recently, inhibition of the Wnt pathway by a soluble GSK3β inhibitor demonstrated increased re-population and presence of pluripotent hematopoetic stem cells in a mouse bone marrow engraftment study (Trowbridge et al. (2006)). Also increased proliferation of a pluripotent stem cell population derived from retina was dependent on the presence of Wnt or GSK3β small molecule inhibitors in in vitro expansion studies (Inoue et al. (2006)). These results suggest Wnt signaling is involved in maintaining a variety of stem cell populations and may be at or near the hierarchical top of pathways that play a role in maintaining stem cell pluripotency.

III) Research in Wnt Pathway Modulators

Since the Wnt pathway has been demonstrated to play a role in the physiology of stem and cancer cells, a great amount of research has been devoted to identifying and understanding the endogenous modulators of this pathway. Three groups of secreted proteins that inhibit Wnt signaling have been identified and recently shown to play a role in modulation of stem cell biology. A family of proteins called secreted frizzled related proteins or SFRPs, have been identified and shown to act as negative modulators of extracellular Wnt signaling by acting to bind to Wnt extracellularly thereby sequestering it and inhibiting it from binding at the Frizzled receptor (Finch et al. (1997)). SFRPs have recently been shown to block the role of Wnt in blocking differentiation in in vitro and in vivo systems (Galli et al. (2006)).

A second family of proteins called dickkopf, or Dkk, also acts to antagonize Wnt signaling extracellularly as well. Dkk was shown to act as a competitive inhibitor of Wnt (Fedi et al. (1999)) and subsequently been shown that it acts upstream of the Wnt/Frizzled receptor formation by inhibiting Wnt co-receptor formation with LRP (Mao et al. (2001)). More recently Dkk has been shown to play a role in maintaining high levels of actively dividing mesenchymal stem cells in multiple labs (Gregory et al. (2003); Etheridge et al. (2004); Byun et al. (2005)). Darwin Prokop and associates have even gone so far as to generate Dkk peptide fragments and determined which sequences of the protein are involved in maintaining pluripotent stem cell expansion (Gregory et al. (2005)).

A third protein that is not a member of either of the SFRP or Dkk families, Wnt inhibitory factor-1 or WIF-1, has also been shown to bind to Wnts with high affinity (Hsieh et al. 1999)). Similar to SFRPs and Dkk WIF-1 has been shown to down-regulate Wnt activity in vivo and in vitro, as well as being demonstrated to have tumor suppressor like qualities in several cancer models. Researchers are beginning to ask if WIF-1 may be fundamentally involved in maintaining pluripotency in stem cells or the stem cell niche.

IV) Summary

The promise of pluripotent cells for regenerative medicine, be they embryonic stem cells or multipotent adult cells harvested from any number of tissues, lies in their ability to self-renew in vitro indefinitely, while retaining their ability to differentiate into specific cell types. To realize this promise, an understanding of the molecular basis of pluripotency is helpful. In regards to the protein inhibitors of the Wnt pathway, what can be said regarding the targets reviewed above? Why would inhibitors of a pathway that is fundamentally involved in maintaining stem cell growth and pluripotency be involved or advantageous biologically? The answers may lie in the fine tuning of Wnt signaling that plays a role in maintaining these specialized cells in an immortalized and pluirpotent state.

In 2004 Brandenberger et al. published a bioinformatic paper on the transcriptome of ES cells in Nature Biotechnology where they compared over 148,000 EST from undifferentiated human embryonic stem cells and three differentiated derivative subpopulations. When the proteins involved in the Wnt pathway were examined, a total of 7 Wnt family members, 7 Frizzled family members and 2 LRPs were found in one or more of the ES cell populations examined. Interestingly one Dkk protein and 2 SFRPs were identified, including statistically significant data from a Fisher Exact T test that both SFRP 1 and 2 were significantly expressed in the undifferentiated human ES cells when no significance was found in any of the Wnts themselves. Similar ES transcriptome analysis by a second group confirmed the finding on SFRP1 (Wei et al. (2005)).

The concept of having a tightly maintained Wnt signaling pathway makes sense in the context of its role as a morphogen in other biological contexts. For example, high levels of Wnt pathway signaling leads to osteogenic differentiation in human MSCs, while at low levels of Wnt signaling in the same cell line, MSCs can be maintained and expanded in an uncommitted state (DeBoer et al. (2004)).

Other pathways involved in maintaining pluripotency of stem cells include, but are not limited to, LIF/STAT3 and BMP4/Id.

Agents that Inhibit GSK-3

One embodiment provides methods of culturing non-embryonic cells, that can differentiate into cell types of more than one embryonic lineage, at high cell density with an agent that inhibits GSK-3, such GSK-3α, GSK-3β or GSK-3β2. In one embodiment, the agent inhibits GSK-3β. Another embodiment provides methods of culturing non-embryonic cells, that can differentiate into cell types of more than one embryonic lineage, at high density, with an agent that has a role in the Wnt signaling pathway. Cells can also be cultured with Wnt protein or β-catenin (e.g., via an expression vector expressing the proteins or by culturing in the presence of the proteins directly). Agents of use in the methods of the invention include, but are not limited to, those agents presented in Table 1 (Meijer, L. et al. (2004)).

TABLE 1
Pharmacological inhibitors of GSK-3

| | | IC50 (μM) | |
|---|---|---|---|
| | | | CDK1-cyclin B |
| Inhibitor | Class | GSK-α and GSK-3β | complex |
| Hymenialdisine | Pyrroloazepine | 0.010 (β) | 0.022 |
| Flavopiridol | Flavone | 0.450 | 0.400 |
| Kenpaullone | Benzazepinone | 0.023 (β) | 0.400 |
| Alsterpaullone | Benzazepinone | 0.004 (α); 0.004 (β) | 0.035 |
| Azakenpaullone | Benzazepinone | 0.018 (β) | 2.000 |
| Indirubin-3′-oxime | Bis-Indole | 0.022 (β) | 0.018 |
| 6-Bromoindirubin-3′- | Bis-Indole | 0.005 | 0.320 |
| oxime (BIO) | | | |
| 6-Bromoindirubin-3′- | Bis-Indole | 0.010 | 63.000 |
| acetoxime | | | |
| Aloisine A | Pyrrolopyrazine | 0.650 | 0.150 |
| Aloisine B | Pyrrolopyrazine | 0.750 | 0.850 |
| TDZD8 | Thiadiazolidinone | 2.000 (β); 7.000 | >100; >10^{a} |
| | | (α/β)^{a} | |
| Compound 12 | Pyridyloxadiazole | 0.390 (β); 8.000 | >10^{a} |
| | | (α/β)^{d} | |
| Pyrazolopyridine 18 | Pyrazolopyridine | 0.018 (α) | Inhibits CDK2-cyclin |
| | | | A (95% at 10 μM) |
| Pyrazolopyridine 9 | Pyrazolopyridazine | 0.022 (α) | Inhibits CDK2-cyclin |
| | | | A (90% at 10 μM) |
| Pyrazolopyridine 34 | Pyrazolopyridine | 0.007 (α) | >10 (CDK2-cyclin |
| | | | A) |
| CHIR98014 | Aminopyrimidine | 0.00065 (α); 0.00058 | 3.700 |
| | | (α/β)^{a} | |
| CHIR99021 | Aminopyrimidine | 0.010 (α); 0.007 (β) | 8.800 |
| (CT99021) | | | |
| CT20026 | Aminopyridine | 0.004 (α/β) | |
| Compound 1 | Pyrazoloquinoxaline | 1.000 | 0.600 |
| SU9516 | Oxindole | 0.330; 0.35 (α/β)^{a} | 0.040; 0.022^{a} |
| | (indolinone) | | |
| ARA014418 | Thiazole | 0.104 (β) | >100 (CDK2 and |
| | | | CDK5) |
| Staurosporine | Bisindolylmaleimide | 0.015; 0.089 | 0.006; 0.008 |
| Compound 5a | Bisindolylmaleimide | 0.018 (β) | 0.24 |
| Compound 29 | Azaindolylmaleimide | 0.034 (β) | >10 |
| Compound 46 | Azaindolylmaleimide | 0.036 (β) | >10 |
| GF109203x | Bisindolylmaleimide | 0.190 (β) | 2.300^{a} |
| (bisindolyl- | | | |
| maleimide I) | | | |
| Ro318220 | Bisindolylmaleimide | 0.003-0.038 (β) | |
| (bisindolyl- | | | |
| maleimide IX) | | | |
| SB216763 | Arylindolemaleimide | 0.034 (α); 0.075 | 0.550^{a} |
| | | (α/β)^{a} | |
| SB415286 | Anilinomaleimide | 0.078 (α); 0.13 (α/β)^{a} | 0.900^{a} |
| I5 | Anilinoarylmaleimide | 0.076 (α); 0.160 (β) | >10 (CDK2-cyclin |
| | | | A) |
| CGP60474 | Phenylamino- | 0.010a | 0.017; 0.0006^{d} |
| | pyrimidine | | |
| Compound 8b | Triazole | 0.280 (β) | >250 (CDK2-cyclin |
| | | | A) |
| TWS119 | Pyrrolopyrimidine | 0.030 (β) | |
| Compound 1A | Pyrazolopyrimidine | 0.016 (β) | |
| Compound 17 | Chloromethyl thienyl | 1.00 (β) | |
| | ketone | | |
| Lithium | Atom (competition | 2000.0 | No effect |
| | with Mg²⁺) | | |
| Beryllium | Atom (competition | 6.00 | Inhibits CDK1 |
| | with Mg²⁺ and ATP) | | |
| Zinc | Atom | 15.00 | No effect |
| | (uncompetitive) | | |

Abbreviations;
CDK1, cyclin-dependent kinase 1;
GSK-3, glycogen synthase kinase 3.
(α) or (β) cited after individual values indicates which specific isoform was tested;
(α/β) cited after individual values indicates that a mixture of isoforms was tested.
The absence of (α), (β) or (α/β) indicates that the study cited did not specify which isoform(s) was tested.
^{a}L. Meijer et al., unpublished.

One embodiment provides a culture method in which non-embryonic cells, that can differentiate into cell types of more than one embryonic lineage, are cultured at high cell density in the presence of a compound of formula (I):

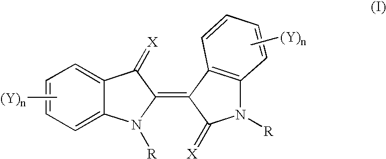

wherein each X is independently O, S, N—OR¹, N(Z), or two groups independently selected from H, F, Cl, Br, I, NO₂, phenyl, and (C₁-C₆)alkyl, wherein R¹ is hydrogen, (C₁-C₆)alkyl, or (C₁-C₆)alkyl-C(O)—;

each Y is independently H, (C₁-C₆)alkyl, (C₁-C₆)alkyl-C(O)—, (C₁-C₆)alkyl-C(O)O—, phenyl, N(Z)(Z), sulfonyl, phosphonyl, F, Cl, Br, or I;

each Z is independently H, (C₁-C₆)alkyl, phenyl, benzyl, or both Z groups together with the nitrogen to which they are attached form 5, 6, or 7-membered heterocycloalkyl;

each n is independently 0, 1, 2, 3, or 4;

each R is independently H, (C₁-C₆)alkyl, (C₁-C₆)alkyl-C(O)—, phenyl, benzyl, or benzoyl; and

wherein alkyl is branched or straight-chain, optionally substituted with 1, 2, 3, 4, or 50H, N(Z)(Z), (C₁-C₆)alkyl, phenyl, benzyl, F, Cl, Br, or I; and

wherein any phenyl, benzyl, or benzoyl is optionally substituted with 1, 2, 3, 4, or 50H, N(Z)(Z), (C₁-C₆)alkyl, F, Cl, Br, or I;

or a salt thereof.

While the compound of formula (I) is depicted above in the Z-double bond configuration, another embodiment provides compounds in the E-double bond configuration or a combination of compounds in the Z- and E-double bond configuration.

Sterioisomers and tautamers of the compounds of formula (I) are also included. Certain organic compounds may exist in two or more tautomeric forms. As referred to herein, the terms “tautomer” or “tautomeric” refer to organic structures in which the carbon and heteroatom connectivities are unchanged, but the disposition of hydrogen atoms in the structures differ. For example, the compound BIO may exist in either of the tautomeric forms as shown below:

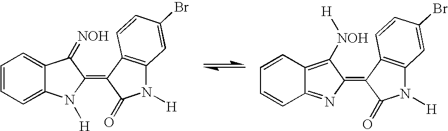

As can be seen, the proton, or hydrogen atom, bonded to the indole nitrogen in the tautomer shown on the left side of the equilibrium is moved to a position on the nitrogen atom of the oxime (hydroxylamine) in the tautomer shown on the right side. Tautomers may or may not be in equilibrium with each other under a given set of conditions. It is understood that when referring to either of the tautomeric structures, the other tautomeric structure is included. This is also true of other organic structures wherein tautomerism is a possibility.

For the compounds useful in the methods of the invention, the following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(R⁸) wherein R⁸ is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Specific and preferred values listed below for radicals, substituents, and ranges are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; heterocycloalkyl and heterocycloalkylalkyl includes the foregoing cycloalkyl wherein the ring optionally comprises 1-2 S, non-peroxide O or N(R⁸) as well as 2-5 carbon atoms; such as morpholinyl, piperidinyl, piperazinyl, indanyl, 1,3-dithian-2-yl, and the like; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be formyl, acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be alkyl substituted with 1 or 20H groups, such as hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 3,4-dihydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl (or its N-oxide), thienyl, pyrimidinyl (or its N-oxide), 1H-indolyl, isoquinolyl (or its N-oxide) or quinolyl (or its N-oxide).

In cases where compounds are sufficiently basic or acidic stable nontoxic acid or base salts may be formed. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium), alkaline earth metal (for example calcium or magnesium) or zinc salts can also be made.

Non-embryonic cells, that can differentiate into cell types of more than one embryonic lineage, can be cultured at high densities in the presence of expansion media with one or more GSK-3 inhibitors at a final concentration (at the time of administration (e.g., at the time of addition to cell culture)) of about 10 nM to about 10 μM. For example, the concentration of the inhibitor can be about 10 nM to about 50 nM, about 50 nM to about 100 nM, about 100 nM to about 200 nM, about 200 nM to about 300 nM, about 300 nM to about 400 nM, about 400 nM to about 500 nM, about 500 nM to about 600 nM, about 600 nM to about 700 nM, about 700 nM to about 800 nM, about 800 nM to about 900 nM, about 900 nM to about 1 μM, about 1 μM to about 2 μM, about 2 μM to about 3 μM, about 3 μM to about 4 μM, about 4 μM to about 5 μM, about 5 μM to about 6 μM, about 6 μM to about 7 μM, about 7 μM to about 8 μM, about 8 μM to about 9 μM, or about 9 μM to about 10 μM. For example, at about 0.25 to about 0.1 to about 1.0 to about 2.0 microM can be employed. Optimal concentration can be routinely determined for each cell type based on routine assays known to those of skill in the art, including, but not limited to, assays regarding pluripotency and replicative ability of the cells.

The cells can be cultured and expanded indefinitely in the presence of a GSK-3 inhibitor or other agent. The inhibitor or other agent is generally added at the time fresh media is added or the cells are passaged (e.g., split); however, inhibitor or other agent can be added at any time during culture of cells.

Additional agents to maintain non-embryonic cells in a pluripotent state (undifferentiated state), include compounds which induce hypoxia (e.g., mimics low oxygen conditions). In one embodiment, compounds which induce hypoxia inhibit prolyl hydroxlase, including but not limited to, the hypoxia inducing factor (HIF) small molecule stabilizer FG0041 (Ivan M., et al. (2002)), 3-carboxy-N-hydroxy pyrollidine (Schlemminger I. et al. (2003)), 3,4 dihydroxybenzoate (Warnecke, C. et al. (2003)). Additional compounds include TGF-β family members, including Cripto and Lefty. Such compounds may be complexed with a GSK-3 inhibitor, including BIO.

### Non-Embryonic Cells

The methods of the invention are applicable to all non-embryonic cells that can differentiate into cell types of more than one embryonic lineage. In one embodiment, the cell is a non-embryonic, non germ, non-embryonic germ cell that can form cell types of two or more embryonic lineages. Such a cell could form cell types of all three embryonic lineages, i.e., endoderm, ectoderm and mesoderm. The cell may express one or more of the genes reported to characterize the embryonic stem cell, i.e., telomerase or Oct-3A. Non-embryonic, non-germ, non-embryonic germ cells that can form cells of more than one primitive germ layer have been described, for example, in U.S. Pat. No. 7,015,037, which is incorporated herein by reference for describing such cells and methods of production.

Non-embryonic cells that can differentiate into cell types of more than one embryonic lineage can be derived from any non-embryonic subject including any organ or tissue, such as umbilical cord, umbilical cord blood, umbilical cord matrix, neural, placenta, bone, brain, kidney, liver, bone marrow, adipose, oogonia, spermatogonia, pancreas, or peripheral blood. For example, non-embryonic cells that can differentiate into cell types of more than one embryonic lineage include non-embryonic stem cells, including but not limited to MAPCs, and other progenitor cells.

The methods and compositions of the invention may also apply to tissue-specific stem cells, such as neural, hematopoietic and mesenchymal; using the compounds of the invention maintains or improves the potency of the cells compared to not using the compounds.

The methods of the invention apply to culturing heterogeneous, as well as substantially homogeneous, populations of cells in the presence of compounds of the invention so that the potency (differentiation capacity) of the population is maintained or enhanced compared to the potency in the absence of the compounds. These populations may contain mixed cell types where cells in the population are of different potencies (e.g., some are committed to a single lineage, others to two lineages, still others to all three lineages). Populations may be restricted to single lineage cells so that all of the cells are endodermal progenitors, for example. Or there could be mixed populations where there are two or more types of single-lineage progenitors, for example, endodermal and mesodermal progenitors.

The methods of the invention may also apply to differentiated cells. For example, the inhibitors of GSK-3 may de-differentiate cells.

### Cell Density

Cells can be cultured at different densities, e.g., cells can be seeded or maintained in the culture dish at different densities or grown to high densities prior to passage. For example, for non-embryonic cells that can differentiate into more than one embryonic lineage, the cells can be seeded or maintained at low or high cell densities. Adherent, non-embryonic cells that can differentiate into more than one embryonic lineage can be seeded or maintained at low densities, including, but not limited to, densities of less than about 2000 cells/cm², including less than about 1500 cells/cm², less than about 1,000 cells/cm², less than about 500 cells/cm², or less than about 200 cells/cm².

Cells of use in the invention can also be seeded or maintained at greater than about 2,500 cells/cm², greater than about 3,000 cells/cm², greater than about 3,500 cells/cm², greater than about 4,000 cells/cm², greater than about 4,500 cells/cm², greater than about 5,000 cells/cm², greater than about 5,500 cells/cm², greater than about 6,000 cells/cm², greater than about 6,500 cells/cm^{,} greater than about 7,000 cells/cm², greater than about 7,500 cells/cm² or greater than about 8,000 cells/cm².

Adherent non-embryonic cells that can differentiate into more than one embryonic lineage can be seeded or maintained at high densities, including, but not limited to, densities of greater than about 8,000 cells/cm², greater than about 10,000 cells/cm², greater about 12,000 cells/cm², greater than about 14,000 cells/cm², greater than about 16,000 cells/cm², greater than about 18,000 cells/cm², greater than about 20,000 cells/cm², greater than about 22,000 cells/cm², greater than about 24,000 cells/cm², greater than about 26,000 cells/cm², greater than about 28,000 cells/cm², greater than about 30,000 cells/cm², greater than about 32,000 cells/cm², greater than about 34,000 cells/cm², greater than about 36,000 cells/cm², greater than about 38,000 cells/cm², greater than about 40,000 cells/cm², greater than about 42,000 cells/cm², greater than about 44,000 cells/cm², greater than about 46,000 cells/cm², greater than about 48,000 cells/cm², greater than about 50,000 cells/cm². For example, the cells can be seeded or maintained at about 8,000 cells/cm² to about 50,000 cells/cm², including, but not limited to, about 8,000 cells/cm² to about 10,000 cells/cm², about 10,000 cells/cm² to about 15,000 cells/cm², about 15,000 cells/cm² to about 20,000 cells/cm², about 20,000 cells/cm² to about 30,000 cells/cm², about 30,000 to about 40,000 cells/cm² or about 40,000 cells/cm² to about 50,000 cells/cm².

Non-adherent human non-embryonic cells that can differentiate into more than one embryonic lineage can be seeded or maintained at low densities, including about 5,000 cells/ml to about 10,000 cells/ml, about 10,000 cells/ml to about 15,000 cells/ml and about 15,000 cells/ml to about 20,000 cells/ml.

Cells of use in the invention can also be seeded or maintained at about 20,000 cells/ml to about 25,000 cells/ml, about 25,000 cells/ml to about 30,000 cells/ml, about 30,000 cells/ml to about 35,000 cells/ml, about 35,000 cells/ml to about 40,000 cells/ml, about 40,000 cells/ml to about 45,000 cells/ml, or about 45,000 cells/ml to about 50,000 cells/ml. These densities are applicable to cells obtained from mammalian sources, including humans.

Non-adherent, non-embryonic cells that can differentiate into more than one embryonic lineage can be seeded or maintained at high densities, for example, at densities, including but not limited to, of greater than about 50,000 cells/ml, greater than about 75,000 cells/ml, greater than about 100,000 cells/ml, greater than about 125,000 cells/ml, greater than about 150,000 cells/ml, greater than about 175,000 cells/ml, greater than about 200,000 cells/ml, greater than about 225,000 cells/ml, greater than about 250,000 cells/ml, greater than about 275,000 cells/ml, greater than about 300,000 cells/ml, greater than about 325,000 cells/ml, greater than about 350,000 cells/ml, greater than about 375,000 cells/ml, greater than about 400,000 cells/ml, greater than about 425,000 cells/ml, greater than about 450,000 cells/ml, greater than about 475,000 cells/ml or greater than about 500,000 cells/ml. For example, the cells can be seeded or maintained at about 50,000 cells/ml to about 500,000 cells/ml, including, but not limited to, about 50,000 cells/ml to about 100,000 cells/ml, about 100,000 cells/ml to about 200,000 cells/ml, about 200,000 cells/ml to about 300,000 cells/ml, about 300,000 cells/ml to about 400,000 cells/ml, about 400,000 cells/ml to about 500,000 cells/ml.

The densities described herein above are applicable to cells obtained from mammalian sources, including humans.

For rodent derived cells, the following densities may be applicable: low cell density including about 50 cells/cm² to about 100 cells/cm², about 100 cells/cm² to about 150 cells/cm², about 150 cells/cm² to about 200 cells/cm², about 200 cells/cm² to about 250 cells/cm², about 250 cells/cm² to about 300 cells/cm², about 300 cells/cm² to about 350 cells/cm², about 350 cells/cm² to about 400 cells/cm², about 400 cells/cm² to about 450 cells/cm², about 450 cells/cm² to about 500 cells/cm².

Cells can also be cultured at about 500 cells/cm² to about 600 cells/cm², about 600 cells/cm² to about 700 cells/cm², about 700 cells/cm² to about 800 cells/cm², about 800 cells/cm² to about 900 cells/cm², about 900 cells/cm² to about 1000 cells/cm². High density can include greater than about 1000 cells/cm².

Essentially, any cell density (seeding density, growth density or maintenance density), low or high up to an including confluent, can be used in the methods of the invention. At each density, the concentration of GSK-3 inhibitors, other agents or culture conditions can be adjusted to obtain the desired result, e.g., maintenance of cell potency (differentiation capacity).

### Multipotent Adult Progenitor Cells (MAPCs)

Human non-embryonic, non-germ, non-embryonic germ cells having the ability to differentiate into cells of multiple primitive germ layers are described in U.S. patent application Ser. Nos. 10/048,757 (U.S. Pat. No. 7,015,037; PCT/US00/21387 (published as WO 01/11011)) and 10/467,963 (PCT/US02/04652 (published as WO 02/064748)), the contents of which are incorporated herein by reference for their description of the cells and production methods. MAPCs have been identified in other mammals and tissues. Murine MAPCs, for example, are also described in PCT/US00/21387 (published as WO 01/11011) and PCT/US02/04652 (published as WO 02/064748). Rat MAPCs are also described in WO 02/064748. In some documents, the cells were termed “MASCs,” an acronym for multipotent adult stem cells. Such cells have also been reported to occur in cord blood, adipose and placenta.

### Compositions

The invention provides a composition comprising non-embryonic cells in combination with at least one GSK-3 inhibitor, wherein said cells can differentiate into cell types of more than one embryonic lineage. Compositions include cells in culture medium. Compositions can be in vitro, ex vivo or in vivo. The invention also provides a method of making a composition comprising admixing non-embryonic cells with at least one GSK-3 inhibitor, and optionally admixing a carrier (e.g., cell culture medium or a pharmaceutically acceptable carrier), wherein said cells can differentiate into cell types of more than one embryonic lineage.

### Isolation and Growth

Methods of MAPC isolation for humans and mouse are described in the art. They are described in PCT/US00/21387 (published as WO 01/11011) and for rat in PCT/US02/04652 (published as WO 02/064748), and these methods, along with the characterization of MAPCs disclosed therein, are incorporated herein by reference.

### General Cell Culture Conditions

Non-embryonic cells can be maintained and expanded in culture medium that is available to the art. Such media include, but are not limited to Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®. Many media are also available as a low-glucose formulation, with or without sodium pyruvate.

Also contemplated is supplementation of cell culture medium with mammalian sera. Sera often contain cellular factors and components that are needed for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements, and bovine embryonic fluid. It is understood that sera can be heat-inactivated at about 55-65° C. if deemed necessary to inactivate components of the complement cascade.

Additional supplements can also be used advantageously to supply the cells with the trace elements for optimal growth and expansion. Such supplements include insulin, transferrin, sodium selenium and combinations thereof. These components can be included in a salt solution such as, but not limited to, Hanks' Balanced Salt Solution® (HBSS), Earle's Salt Solution®, antioxidant supplements, MCDB-201® supplements, phosphate buffered saline (PBS), ascorbic acid and ascorbic acid-2-phosphate, as well as additional amino acids. Many cell culture media already contain amino acids, however some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine. It is well within the skill of one in the art to determine the proper concentrations of these supplements.

Antibiotics are also typically used in cell culture to mitigate bacterial, mycoplasmal and fungal contamination. Typically, antibiotics or anti-mycotic compounds used are mixtures of penicillin/streptomycin, but can also include, but are not limited to, amphotericin (Fungizone®), ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin and zeocin.

Hormones can also be advantageously used in cell culture and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine and L-thyronine.

Lipids and lipid carriers can also be used to supplement cell culture media, depending on the type of cell and the fate of the differentiated cell. Such lipids and carriers can include, but are not limited to, cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

Also contemplated is the use of feeder cell layers. Feeder cells are used to support the growth of fastidious cultured cells, including stem cells. Feeder cells are normal cells that have been inactivated by γ-irradiation. In culture, the feeder layer serves as a basal layer for other cells and supplies cellular factors without further growth or division of their own (Lim, J. W. and Bodnar, A., (2002)). Examples of feeder layer cells are typically human diploid lung cells, mouse embryonic fibroblasts, Swiss mouse embryonic fibroblasts, but can be any post-mitotic cell that is capable of supplying cellular components and factors that are advantageous in allowing optimal growth, viability and expansion of cells. In many cases, feeder cell layers are not necessary to keep the ES cells in an undifferentiated, proliferative state, as leukemia inhibitory factor (LIF) has anti-differentiation properties. Therefore, supplementation with LIF could be used to maintain non-embryonic cells in an undifferentiated state. Additionally, a GSK-3 inhibitor may be used to maintain non-embryonic cells in an undifferentiated state.

Cells in culture can be maintained either in suspension or attached to a solid support, such as extracellular matrix components and synthetic or biopolymers. Cells sometimes require additional factors that encourage their attachment to a solid support, such as type I, type II and type IV collagen, concanavalin A, chondroitin sulfate, fibronectin, “superfibronectin” and fibronectin-like polymers, gelatin, laminin, poly-D and poly-L-lysine, thrombospondin and vitronectin.

The maintenance conditions of non-embryonic cells can also contain cellular factors that allow the non-embryonic cells, such as MAPCs, to remain in an undifferentiated form. It is advantageous under conditions where the cell must remain in an undifferentiated state of self-renewal for the medium to contain epidermal growth factor (EGF), platelet derived growth factor (PDGF), leukemia inhibitory factor (LIF; in selected species), a GKS-3 inhibitor or combinations thereof. It is apparent to those skilled in the art that supplements that allow the cell to self-renew but not differentiate should be removed from the culture medium prior to differentiation.

Cells can benefit from co-culturing with another cell type. Such co-culturing methods arise from the observation that certain cells can supply yet-unidentified cellular factors that allow the cell to differentiate into a specific lineage or cell type. These cellular factors can also induce expression of cell-surface receptors, some of which can be readily identified by monoclonal antibodies. Generally, cells for co-culturing are selected based on the type of lineage one skilled in the art wishes to induce, and it is within the capabilities of the skilled artisan to select the appropriate cells for co-culture.

Methods of identifying and subsequently separating differentiated cells from their undifferentiated counterparts can be carried out by methods well known in the art. Cells that have been induced to differentiate can be identified by selectively culturing cells under conditions whereby differentiated cells outnumber undifferentiated cells. Similarly, differentiated cells can be identified by morphological changes and characteristics that are not present on their undifferentiated counterparts, such as cell size, the number of cellular processes (i.e., formation of dendrites or branches), and the complexity of intracellular organelle distribution. Also contemplated are methods of identifying differentiated cells by their expression of specific cell-surface markers such as cellular receptors and transmembrane proteins. Monoclonal antibodies against these cell-surface markers can be used to identify differentiated cells. Detection of these cells can be achieved through fluorescence activated cell sorting (FACS) and enzyme-linked immunosorbent assay (ELISA). From the standpoint of transcriptional upregulation of specific genes, differentiated cells often display levels of gene expression that are different from undifferentiated cells. Reverse-transcription polymerase chain reaction (RT-PCR) can also be used to monitor changes in gene expression in response to differentiation. In addition, whole genome analysis using microarray technology can be used to identify differentiated cells.

Accordingly, once differentiated cells are identified, they can be separated from their undifferentiated counterparts, if necessary. The methods of identification detailed above also provide methods of separation, such as FACS, preferential cell culture methods, ELISA, magnetic beads, and combinations thereof. A preferred embodiment of the invention envisions the use of FACS to identify and separate cells based on cell-surface antigen expression.

Effective atmospheric oxygen concentrations of less than about 10%, including about 3% to about 5% O₂, can be used at any time during the isolation, growth and differentiation of cells in culture. Cells may also be cultured in the presence of beta mercaptoethanol (BME), for example, at initial culture concentrations of about 0.1 mM.

### Use of Non-Embryonic Cells

Non-embryonic cells, that can differentiate into cell types of more than one embryonic lineage, grown in the presence of a GKS-3 inhibitor can be used in preclinical, such as in large animal models of disease, and clinical, such as therapeutic, settings (use of MAPCs isolated from humans and mice are described in PCT/US0021387 (published as WO 01/11011) and from rat in PCT/US02/04652 (published as WO 02/064748), and these are incorporated herein by reference).

Non-embryonic cells that can differentiate into cell types of more than one embryonic lineage can be used to treat essentially any injury or disease, particularly a disease associated with pathological organ or tissue physiology or morphology which is amenable to treatment by transplantation in any mammalian species, preferably in a human subject. Thus, non-embryonic cells that can differentiate into cell types of two or more embryonic lineages or progeny derived therefrom can be administered to treat diseases amendable to cell therapy.

For example, non-embryonic cells that can differentiate into cell types of more than one embryonic lineage have utility in the repopulation of organs, either in a self-renewing state or in a differentiated state compatible with the organ of interest. They have the capacity to replace cell types that have been damaged, died, or otherwise have an abnormal function because of genetic or acquired disease. Or they may contribute to preservation of healthy cells or production of new cells in a tissue.

Additionally, non-embryonic cells that can differentiate into cell types of more than one embryonic lineage or differentiated progeny derived therefrom can be genetically altered ex vivo, eliminating one of the most significant barriers for gene therapy. For example, non-embryonic cells that can differentiate into cell types of more than one embryonic lineage can be extracted and isolated from the body, grown in culture in the undifferentiated state or induced to differentiate in culture, and genetically altered using a variety of techniques, especially viral transduction. Uptake and expression of genetic material is demonstrable, and expression of foreign DNA is stable throughout development.

### EXAMPLES

The following examples are provided in order to demonstrate and further illustrate certain embodiments and aspects of the present invention and are not to be construed as limiting the scope thereof.

### Example 1

### Synthesis of BIO

3-Indolyl acetate (0.18 g, 1.0 mmol) and Na₂CO₃ (0.53 g, 5.0 mmol) were added to a solution of 6-bromo-isatin (0.23 g, 1.0 mmol) in anhydrous methanol (5 mL) at 0° C. The reaction mixture was stirred overnight at room temperature. The precipitate that formed during the reaction was filtered and washed with H₂O (20 mL) to give 0.2 g (59% yield) of the desired intermediate, which was used without further purification.

The intermediate from the first reaction (0.2 g, 0.59 mmol) and hydroxylamine hydrochloride (45 mg, 0.65 mmol) were dissolved in pyridine (6 mL) and stirred overnight at 60° C. The reaction solution was concentrated via rotary evaporation, and the crude product was purified via preparatory LC-MS. MS calculated for C₁₆H₁₁BrN₃O₂+H: 356, observed: 356.

### Example 2

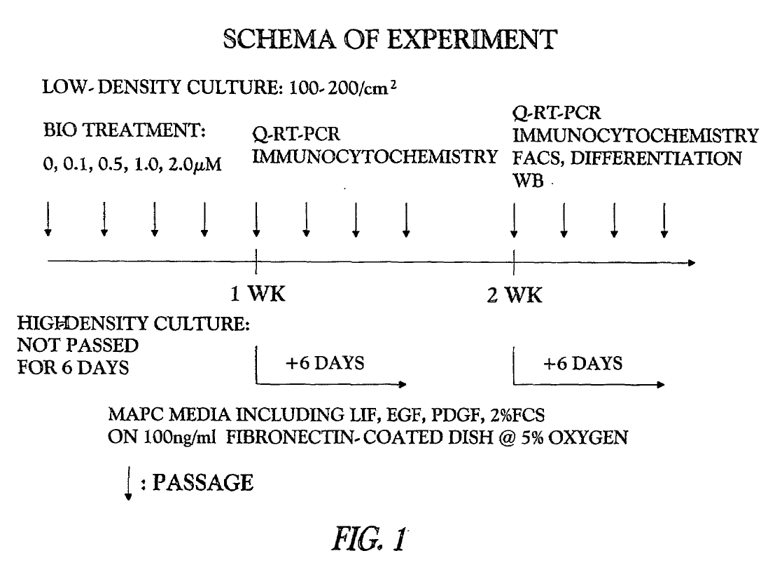

The WNT/β-catenin pathway plays a role in the maintenance of the undifferentiated state of human embryonic stem cells. It was investigated herein if β-catenin activation, by inhibiting GSK3β with BIO, affects the differentiation status of MAPCs. FIG. 1 provides a schematic of the experimental procedure.

### Materials Methods

Cell Culture—Murine MAPC cell lines were established from GFP transgenic C57BL/Ka-Thy1.1 newborn mice bone marrow cells as described in Jiang, Y. et al. (2002)). Murine MAPCs were cultured +/−0.1-2 microM BIO (ranging from 0.1, 0.5, 1.0 or 2.0 μM) in 60% DMEM-LG (Gibco BRL (Gaithersburg Md.)), 40 MCDB-201 with 1×SITE, 0.2×LA-BSA, 0.2 g/l BSA, 0.1 mM ascorbic acid 2-phosphate, 0.1 mM beta mercaptoethanol (Sigma (St. Louis, Mo.)), 100 U penicillin, 1000 U streptomycin (Gibco), 1000 U/ml LIF (Chemicon (Temecula, Calif.), 10 ng/ml mEGF (Sigma), 10 ng/ml hPDGF-BB (R&D Systems (Minneapolis, Minn.)), 2% fetal calf serum (FCS; Hyclone Laboratories (Logan, Utah)) on human 10 ng/cm² fibronectin (Sigma)-coated dishes (Nunc (Rochester, N.Y.)) at 5% CO₂ and 5% O₂. Cells were plated at low density, about 100 cells/cm² and cells were split approximately every two days, or high confluency (similar to the confluency that embryonic stem cells are cultured at (about 100,000 to 500,000 cells per 10 cm dish or about 60% to 80% confluent). Rat MAPCs were established in a similar fashion. One to two weeks after treatment with BIO, several of the cultures were kept growing for four to six days in the same dishes.

Immunohistochemistry—Cells were fixed with 4% paraformaldehyde (Sigma) at room temperature for 15 minutes (min) and incubated sequentially for 30 min each with primary antibodies and DAB. For nuclear ligands, cells were permeabilized with 0.1 M Triton X-100 (Sigma) for 10 min. Slides were washed with PBS (Cellgro) and 1% BSA (Sigma) between each step. Anti-Oct-4 antibody (1:200) was from Santa Cruz; anti-β-catenin antibody (1:150) was from eBiosciences; anti-unphosphorylated β-catenin antibody (1:1000) was from Upstate and anti-E-cadherin antibody (1:400) was from R&D Systems.

Quantitative RT-PCR—RNA was extracted from MAPCs treated with or without BIO weekly by using RNeasy minikit (Qiagen (Valencia, Calif.)). Contaminating DNA was eliminated by DNase treatment (Invitrogen (Carlsbad, Calif.)). mRNA was reverse-transcribed, cDNA underwent 40 cycles of amplification (ABI PRISM 7700, Perkin-Elmer/Applied Biosystems (Foster City, Calif.)) with reaction conditions of 40 cycles of a two step PCR (95° for 15 min and 60° for 60 min) after an initial denaturation (95° for 10 min) with 1 μl of DNA solution (a mixture of dATP, dCTP, dGTP and dTTP) and 1×SYBER green PCR master mix reaction buffer (Applied Biosystems). Controls consisted of amplifications without reverse transcriptase and reaction without addition of DNA template. The authenticity and size of PCR products were confirmed by melting curve analysis. mRNA were normalized by using GAPDH as housekeeping gene and compared with levels (control) in murine embryonic stem cells (for Oct-4, Nanog, Rex-1, Utf-1, Sox-2 and E-Ras), murine midbrain (for Sox 1 and Nestin) and universal reference total RNA (BD Clontech (Mountain View, Calif.)) (for AFP, Albumin, CK19, vWF, CD31, VE-Cadherin).

Flow cyometry—Two or four weeks after treatment with BIO, murine low-density MAPCs were stained with PE-conjugated anti-CD44 (1:50), anti-Sca-1 (1:40), anti-CD34 (1:20), anti-Thy-1.1 (1:160), anti-H-2 K^{b} (1:40), anti-1-A^{b} (1:40), APC-conjugated anti-c-kit (1:50), biotinylated anti-CD9 (1:80), anti-Lineage cocktail (CD3e, B220, Mac-, Gr-1, Ter-119; 1:40), anti-CD45.2 antibodies followed by streptavidin-APC (1:20) (BD Pharmingen (San Diego, Calif.)) and purified anti-SEEA-1 (1:5) (Chemicon (Temecula, Calif.)) followed by APC conjugated anti-mouse IgM, anti-CD49f (1:20), anti-EpCAM (1:20) (BD Pharmingen), anti-E-cadherin (1:5) (R&D Systems), followed by APC-conjugated goat anti-F(ab′)2 rat IgG antibody (Jackson ImmunoResearch (West Grove, Pa.)).

Two weeks after treatment with BIO, rat MAPCs were stained with FITC-conjugated anti-CD44 (1:50), anti-CD11b/c (1:50), PE-conjugated anti-CD45 (1:20), PerCP-conjugated Thy-1.1 (1:10) and biotinylated-anti-RT1A (1:50), anti-RT1B (1:50), anti-CD31 (1:50) antibodies followed by streptavidin-PE staining (1:50), which were all purchased from BD Pharmagin. Cells were incubated with antibodies with 2% FCS for 30 min at 4° C. Cells were washed, resuspended in 200 μl PBS and 2% FCS. Data acquisition and analysis were performed with FACS Calibur and Cell Quest Pro software, respectively (Becton Dickinson).

### Results

BIO caused a dose-dependent clustering of MAPC from 4 days after treatment at both cell densities. FACS phenotype of low-density MAPCs was not affected by BIO: MHC-I/II, CD45, hematopoietic lineage antigen (CD3e, B220, Gr-1, Mac-1, Ter-119), and CD44 negative; while c-kit, CD9, E-cadherin, EPCAM, CD49f positive. Nanog mRNA was not detected in any of the cell populations, and BIO did not affect UTF1 or E-Ras expression.

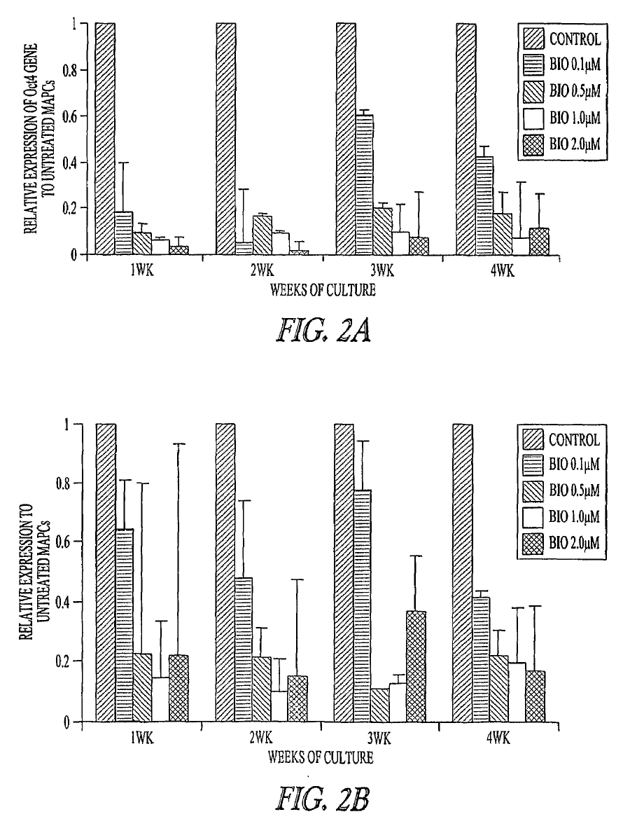

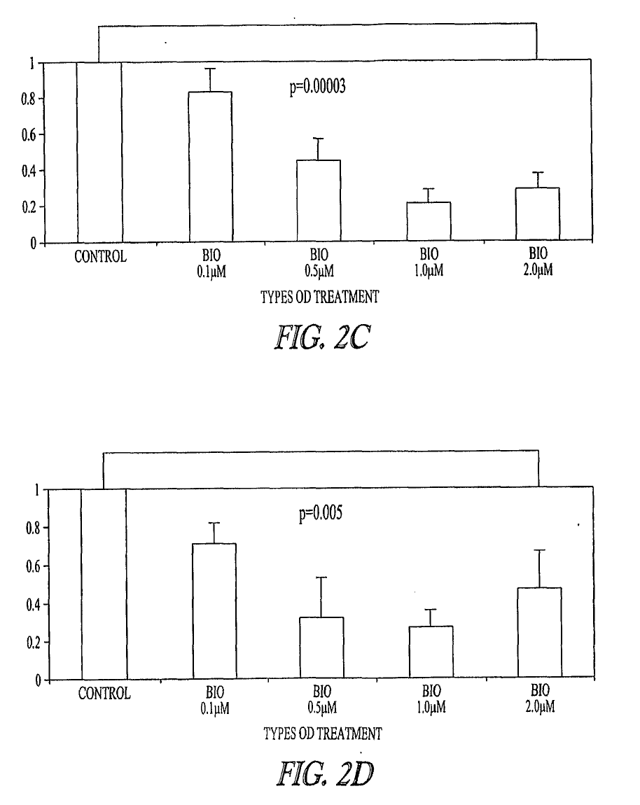

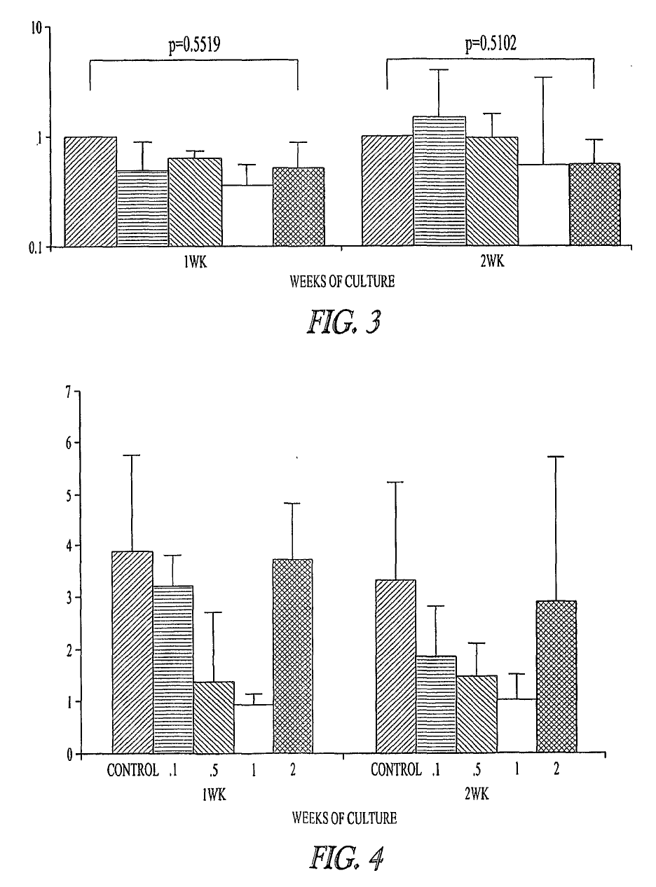

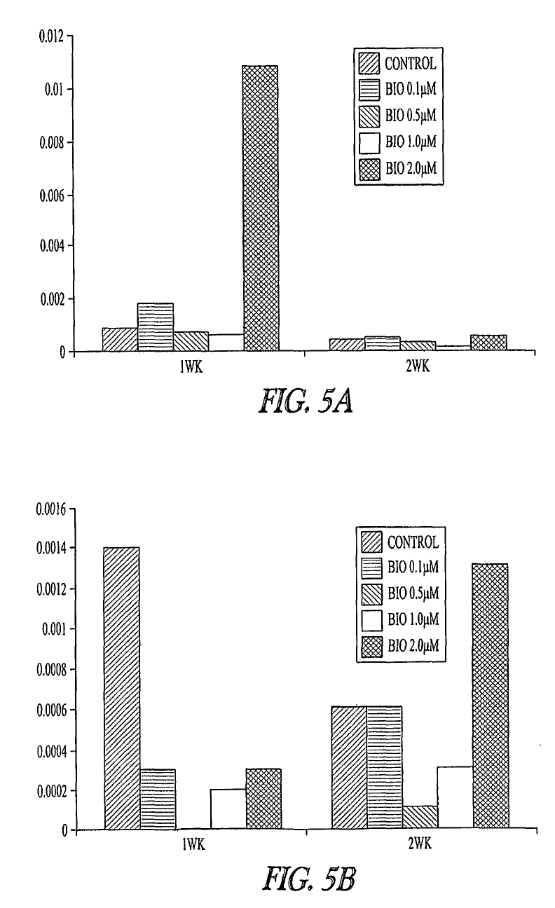

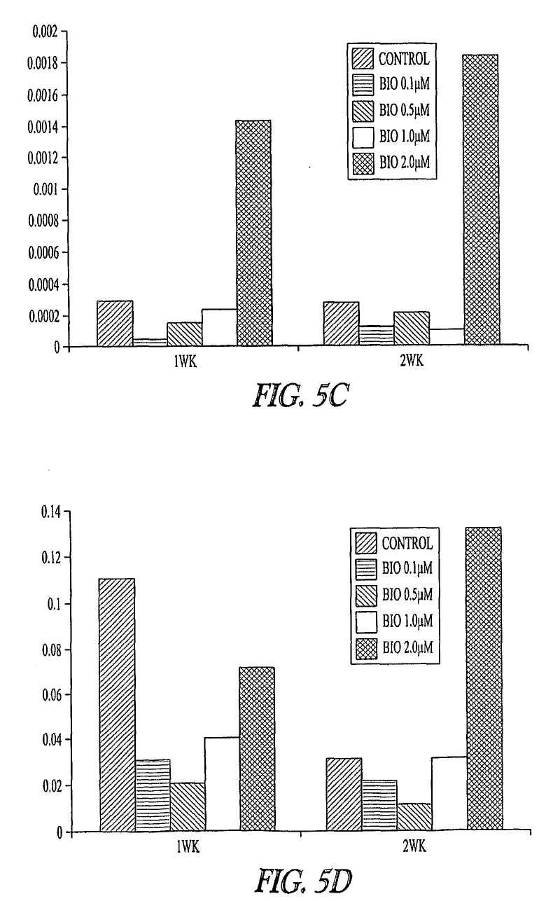

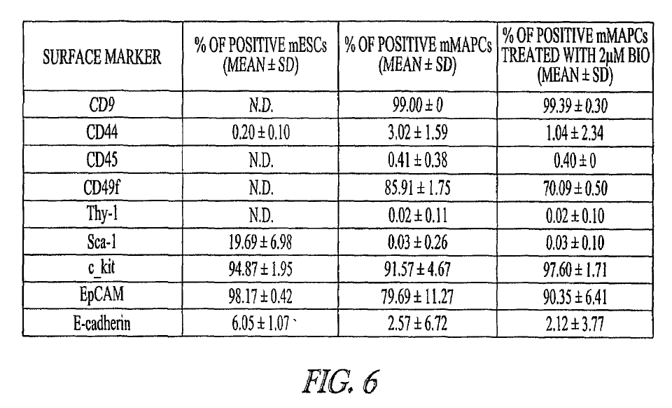

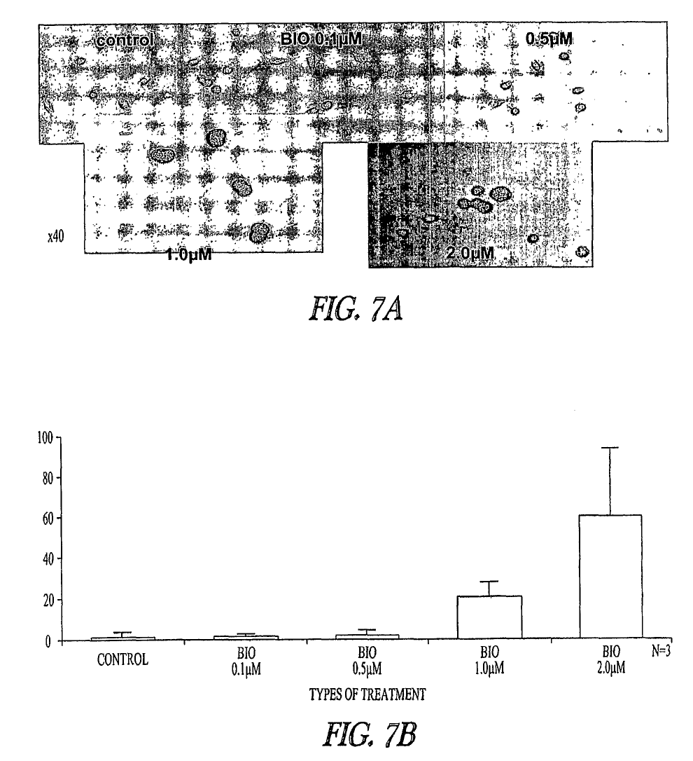

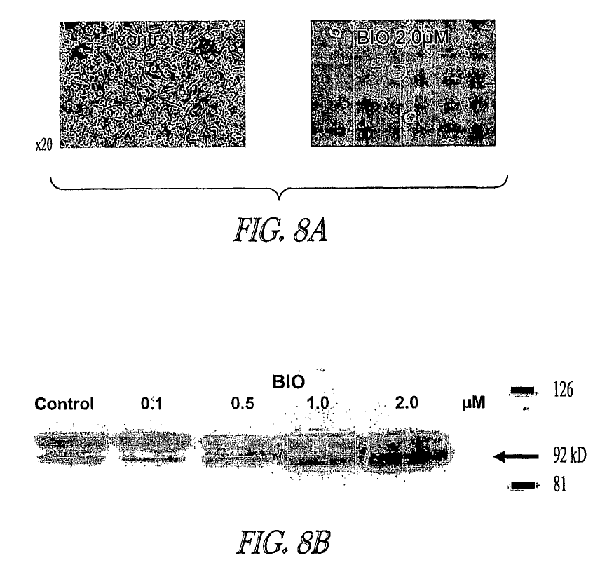

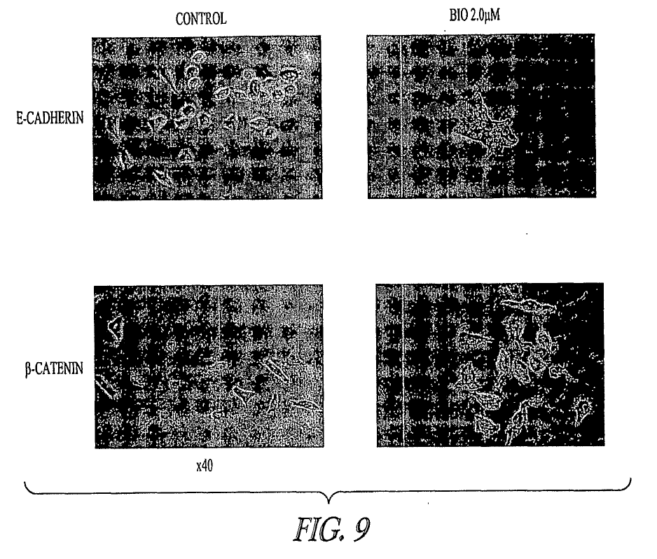

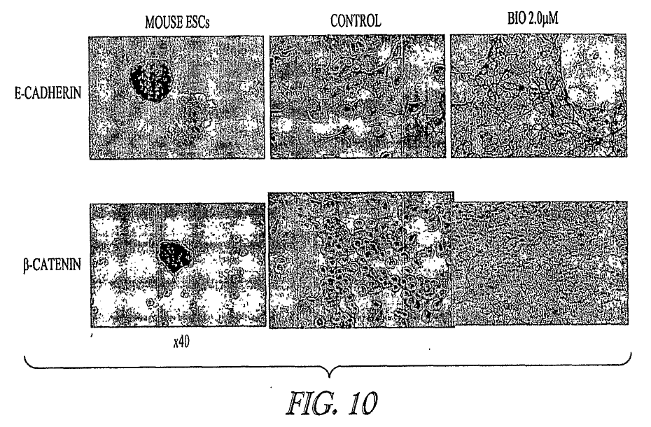

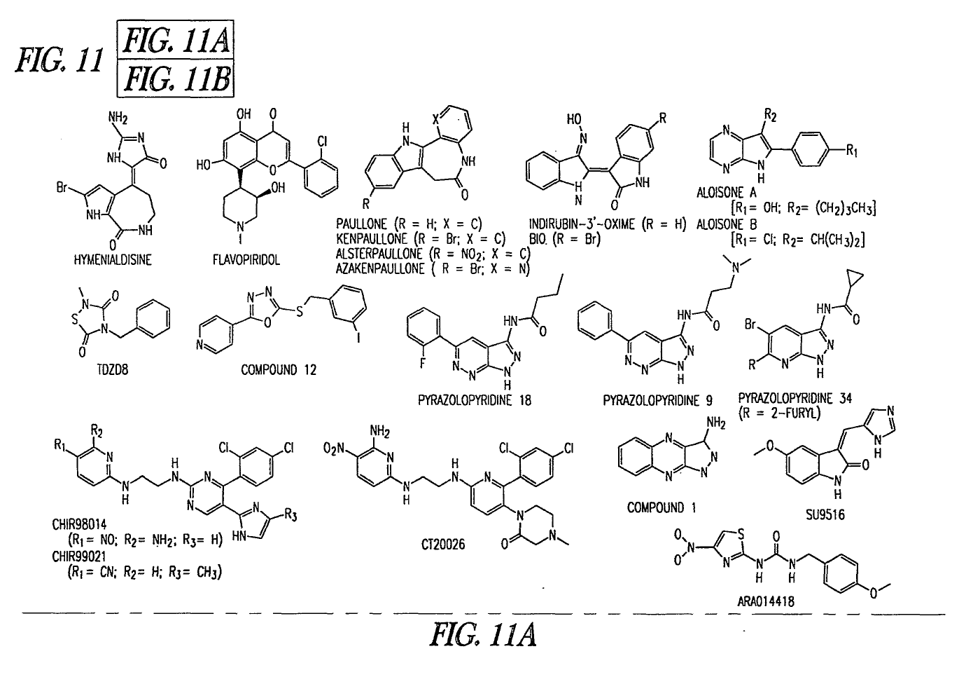

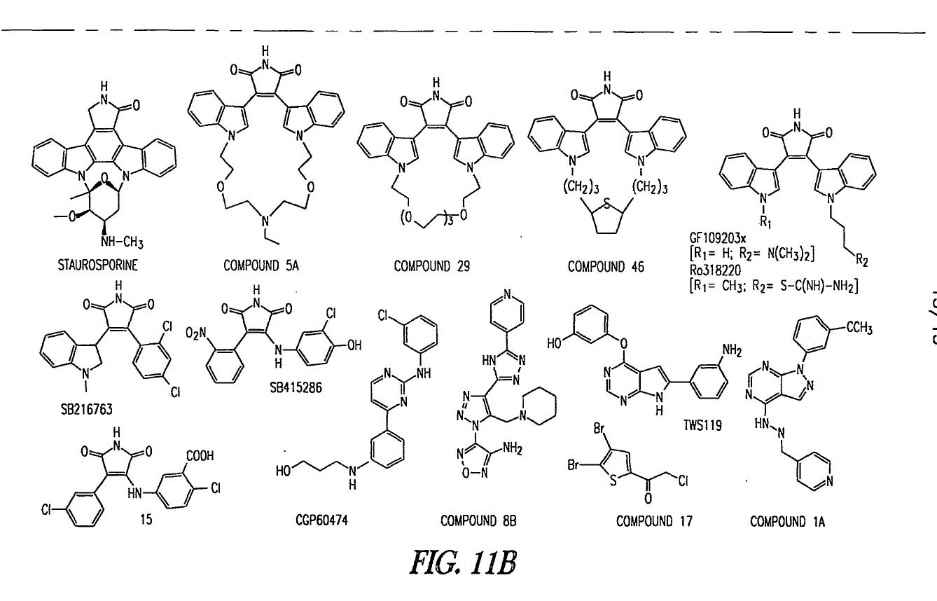

However, Oc-t4 (FIG. 2) and Rex-1 (FIG. 2) mRNA levels decreased in a BIO dose-dependent manner in MAPCs maintained at low density. Low concentration of BIO also resulted in MAPCs, maintained at low density, which had higher levels of endothelial specific genes (for example, vWF (FIG. 1)), while high concentration of BIO resulted in MAPCs, maintained at low density, which had higher levels of AFP (FIG. 4) and Sox1 (FIG. 5). However, Oct-4, β-catenin and E-cadherin proteins were highly expressed in clusters of low-density MAPCs induced by 2 microM BIO.

Surprisingly, when MAPCs were maintained at high density (ESC densities), MAPCs treated with 1 or 2 microM BIO contained large dense MAPC clusters that expressed very high levels of Oct-4, β-catenin and E-cadherin protein.

Thus, BIO, and other GSK-3β inhibitors (including, but not limited to, other indirubins), can allow culture of non-embryonic cells, that can differentiate into more than one embryonic lineage, at high density, such as ESC-like density, without loss of Oct-4. Therefore, non-embryonic cells, that can differentiate into more than one embryonic lineage, can be grown at high densities and remain pluripotent. This use of BIO, and other GSK-3β inhibitors, can therefore aid in large-scale cell culture of non-embryonic cells that can differentiate into more than one embryonic lineage, such as MAPCs.

Additionally, BIO, and other GSK-3β inhibitors, including indirubins, can induce lineage commitment of low-density culture of non-embryonic cells that can differentiate into more than one embryonic lineage. For example, low density cultures of MAPCs treated with concentrations of BIO, and other GSK-3β inhibitors, can induce differentiation of the MAPCs to endothelial cells.

### BIBLIOGRAPHY

Abe, A., et al., J. Virol. 1998; 72: 6159-6163.
Akimenko, M. A., J Neurosci 1994; 14:3475-86.
Alter B P, Cancer Genet Cytogenet. 1992; 58:206-8; discussion 209.
Alvarez-Dolado M., et al. Nature. 2003; 425:968-973.
Ambrosetti, D., et al., Mol. Cell. Biol. 1997, 17:6321-9.
American Cancer Society. Cancer Facts and Figures-2004. http://www.cancer.org/docroot/PRO/content/PRO_{—}1_{—}1_Cancer_Statistics_{—}2004_presentation.asp?
American Diabetes Association. Direct and Indirect Costs of Diabetes in the U.S. http://www.diabetes.org/main/info/facts/facts_costs.jsp
American Heart Association. Heart Disease and Stroke Statistics-2004 Update. http://www.americanheart.org/presenterjhtml?identifier=1200026
Aranguren X L, et al., The arterial and venous potential of human MAPC and AC133 derived endothelial cells is modulated by activation of notch and patched ligands. Keystone Symposium on stem cells. 2005.
Aubert et al. Nature Biotechnology. 2002; 20:1240-45.
Babcook, et al., Proc. Natl. Acad. Sci. (USA). 1996; 93: 7843-7848.
Bain, et al., Biochem J, (2003); 371:199-204.
Barker J N and Wagner J E. Nature Reviews Cancer. 2003; 3:526-532.
Barker J N et al., Blood. 2003; 102:1915-1919.
Barker J N et al., Blood. 2004; ePub.
Barnett M J et al., Blood. 1994; 84:724-732.
Basch, et. al., J. Immunol. Methods. 1983; 56:269.
Batinic, D., et al., Bone Marrow Transplant. 1990; 6(2):103-7.
Ben-Shushan E et al., Mol Cell Biol. 1998; 18:1866-1878.
Bertrand J Y et al., Proc Natl Acad Sci USA. 2005; 102:134-139.
Bertrand, et al., J. Mol. Biol., (2003); 333:393-407.
Bhardwaj G et al. Nat Immunol. 2001; 2:172-180.
Bhat, et al., J Biol. Chem., (2003); 278:45937-45945.
Bhatia M et al., Nat. Med. 1998; 4:1038-1045.
Bhatia R et al., Blood. 1995; 85:3636-3645.
Bhatia R et al., J Clin Invest. 1994; 94:384-391.
Bhatia R et al., Blood. 2003; 101:4701-4707.
Bierhuizen, M. et al., Blood. 1997; 90(9):3304-3315.
Bird, et al., Science. 1988; 242:423-426.
Bittira B. et al. Eur J Cardiothorac Surg. 2003; 24:393-398.
Bjorklund L Mea. Proc Natl Acad Sci USA. 2002; 99:2344-2349.
Boeuf, H., et al. J. Cell Biol. 1997; 138:1207-17.
Borue X, et al. Am J Pathol. 2004; 165:1767-1772.
Brandenberger et al. Nature Biotechnology. 2004; 22:707-16.
Bravo, G., and J. Heath. EMBO 2000; 19:2399-2411.
Bredenbeek, P. J., et al. J. Virol. 1993; 67:6439-6446.
Brehm, A., et al. Molecular and Cellular Biology 1997; 17:154-62.
Brice, G. T., et al., J. Acquir Immune Defic Syndr Hum Retrovirol. 1998; 19:210-220.
Buckley S et al., Stem Cells. 2004.
Byun et al. J. Clin. Path. 2005; 58:515-19.
Cadigan, K. and R Nusse. Genes Dev. 1997; 11:3286-3305.
Cai, J. and M. S. Rao. Neuromolecular Medicine 2002; 2:233-49.
Cai Z. H., et al., Artif Organs. 1988; 12(5):388-93.
Camargo F D et al., J Clin Invest. 2004; 113:1266-1270.
Carella A M et al., Blood Rev. 1997; 11:154-159.
Cerdan C et al., Blood. 2004; 103:2504-2512.
Chambers I et al., Cell. 2003; 113:643-655.
Chang, P., et al., Trends in Biotech. 1999; 17:78-83.
Chang, T. M., Artif Organs. 1992; 16(1):71-4.
Chen, S., et al. JACS 2003; 126:410-11.
Chenn, A. and C. Walsh. Science 2002; 297:365-9.
Choi K et al., Biochem Cell Biol. 1998; 76:947-956.
Choi K et al., Development. 1998; 125:725-732.
Clackson et al. Nature. 1991; 352:624-628.
Clarke, Science. 2000; 288:1660-3.
Clavel C et al., Isolation and Characterization of Multipotent Adult Progenitor Cells from Cynomologus Monkey. Keystone Symposium on stem cells. 2005.
Clothia et al., J. Mol. Biol. 11985; 186:651-66, 1985.
Coghlan, et al., Chem. Biol., (2000); 7:793-803.
Coligan, et al., Current Protocols in Immunology, (1991 and 1992).
Conde, et al., J. Med. Chem., (2003); 46:4631-4633.
Dao M A and Nolta J A. Int J Mol. Med. 1998; 1:257-264.
Darnell, J. (1996) PNAS 93:6221-4.
Davidson, B. L., et al., Nature Genetics. 1993; 3:219-223.
Davies, et al., Biochem. J, (2000); 351:95-105.
DeBoer et al. Tissue Engineering. 2004; 10:393-401.
Deisseroth A B et al., Blood. 1994; 83:3068-3076.
Derynck, R. and X. H. Feng. Biochim. Biophys. Acta 1997; 1333:F105-F150.
Derynck, R. and Y. Zhang. Nature 2003; 425: 577-84.
Ding, et al., Proc. Natl. Acad. Sci. U.S.A., (2003); 100:7632-7637.
Douglas, J., et al. Nature Biotech. 1999; 17:470-475.
Douglas, J. et al., Hum. Gene Ther. 1999; 10(6):935-945.
Drukker M, et al. Proc Natl Acad Sci USA. 2002; 99:9864-9869.
Dull, T., et al., J. Virol. 1998; 72:8463-8471.
Dyer M A et al., Development. 2001; 128:1717-1730.
Eastman, Q. and R. Grosschedl. Curr. Opin. Cell Biol. 1999; 11:233-40.
Eckfeldt C E et al., PLoS Biology. 2004.
Etheridge et al. Stem Cells 2004; 22:849-60.
Evans, M. and M. Kaufman. Nature 1981; 292:154-6.
Faloon P et al., Development. 2000; 127:1931-1941.
Fedi et al. J. Biol. Chem. 1999; 274:19465-72.
Ferrari, Science. 1998; 279:528-30.
Finch et al PNAS. 1997; 94:6770-5.
Foerst-Potts, L. Dev Dyn 1997; 209:70-84.
Fortunel, N., et al. Stem Cells 2000; 18:102-11.
Frolov, I., et al. (Proc. Natl. Acad. Sci. USA. 1996; 93:11371-11377.
Galli et al. Developmental Dynamics 2006; 235:681-90.
Gambacorti-Passerini C B et al., Lancet Oncol. 2003; 4:75-85.
Gilboa, L., et al. Mol. Biol. Cell 2000; 11:1023-35.
Gluckman E. Stem Cells 1993; 11 Suppl 2:180-3.
Grant M B et al., Nat. Med. 2002; 8:607-612.
Gregory et al. J. Biol. Chem. 2003; 278:28067-78.
Gregory et al. J. Biol. Chem. 2005; 280:2309-23.
Guardiola P, et al., Blood. 2000; 95:422-9.
Guinan E C et al., J Pediatr 1994; 124:144-50.
Gunsilius E et al., Lancet. 2000; 355:1688-1691.
Gupta P et al., Blood. 1996; 87:3229.
Gupta P et al., Blood. 1998; 92:4641-4651.
Gussoni, Nature. 1999; 401:390-4.
Hale et al. Journal of Bone and Mineral Research. (2005); 20(9):S365.
Hansis, C., et al. Mol. Hum. Reprod. 2000; 6:999-1004.
Harraz M et al., Stem Cells. 2001; 19:304-312.
Harris R G et al., Science. 2004; 305:90-93.
Harvey K and Dzierzak E. Stem Cells. 2004; 22:253-258.
Hematti P et al., PLOS Biology. 2004; 2:e243.
Hemmati-Brivanlou A et al., Dev Genet. 1995; 17:78-89.
Hers, et al., FEBS Lett., (1999); 460:433-436.
Hofmann, C., et al. J. Virol. 1999; 73:6930-6936.
Hogan C J et al., Blood. 1997; 90:85-96.
Hollinger et al., Proc. Natl. Acad. Sci. USA. 1993; 906444-6448 (1993).
Holmes, et al., J. Immunol. 1997; 158:2192-2201.
Holyoake T L et al., Exp Hematol. 1999; 27:1418-1427.
Horrocks, C., et al. Curr Opin Drug Discov Devel. 2003; 6:570-5.
Howe C W S, Radde-Stepanick T. Hematopoietic Cell Donor Registries. In: Thomas E D, Blume, Karl G. and Forman, Stephan J., ed. Hematopoietic Cell Transplantation. Vol. 2. Malden, M A: Blackwell Sciences; 1999:503-512.
Hsieh et al. Nature. 1999; 398:431-36.
Hurley R W et al., J Clin Invest. 1995; 96:511-521.
Ilouz, et al., Biochim. Biophys. Res. Commun., (2002); 295:102-106.
Inoue, et al., Stem Cells 2006; 24:95-104.
Itoh, S., et al. (2000) Eur. J. Biochem. 267:6954-67.
Ivan, M., et al., PNAS 2002; 99:13459-13464.
Jackson, PNAS USA. 1999; 96:14482-6.
Jahagirdar, B. N., et al. Exp Hematol. 2001; 29(5):543-56.
Jiang Y et al., Blood. 2000; 95:846-854.
Jiang Y et al., Proc Natl Acad Sci USA. 2000; 97: 10538-10543.
Jiang Y H D et al., Proc Natl Acad Sci USA. 2003; 100 Suppl 1:11854-11860.
Jiang Y, et al., Exp Hematol. 2002b; 30:896-904.
Jiang Y, et al., Nature. 2002; 418:41-49.
Jiang Y, et al., Nature. 2002a; 418:41-49.
Jiang Y, et al., Proc Natl Acad Sci USA. 2003; 100 Suppl 1:11854-11860.
Johnston, S. A., et al., Genet. Eng. (NY) 1993; 15: 225-236.
Jones et al., Nature. 1986; 321:522-525.
Kafri, T., et al., J. Virol. 1999; 73:576-584.
Kannagi, R. EMBO J. 1983; 2:2355-61.
Kantarjian H M et al., Blood. 2003; 101:97-100.
Kaufman D S et al., Proc Natl Acad Sci USA. 2001; 98:10716-10721.
Kawada H, et al. Blood. 2004; 104:3581-3587.
Kehat, I., et al. (2001) J. Clin. Invest. 108:407-14.
Kemahli S et al., Br J Haematol 1994; 87:871-2.
Kim, J. H., et al. Nature 2002; 418:50-6.
Kirchhof, N., et al. Biology of Reproduction 2000; 63:1698-1705.
Klein, et al., Proc. Natl. Acad. Sci. U.S.A., (1996); 93:8455-8459.
Kogler G et al., J Exp Med. 2004; 200:123-135.
Kohler & Milstein, Nature. 1975; 256:495.
Kohli-Kumar M et al., Blood. 84:2050-4, 1994.
Korinek, V. et al. Nature Genetics 1998; 19:1-5.
Korinek, V. et al. Science 1997; 275:1784-7.
Krause D S, et al. Cell. 2001; 105:369-377.
Kulkarni et al. J. Bone Miner. Res. (2006);21(6):910-20.
Kunick, et al., Bioorg. Med. Chem. Lett., (2004); 14:413-416.
Kuo, et al., Cancer Res., (2001); 61:6170-6177.
Kuo, et al., J. Med. Chem., (2003); 46:4021-4031.
Kusadasi N et al., Leukemia. 2002; 16:1782-1790.
Kyba M et al., Cell. 2002; 109:29-37.
Kyba M et al., Proc Natl Acad Sci USA. 2003; 100, Suppl 1:11904-11910.
Lagasse E, et al. Nat. Med. 2000; 6:1229-1234.
Lanier L L. “NK Cell Recognition.” Annu Rev Immunol. 2004.
Laquerre, S., et al. J. Virol. 1998; 72:9683-9697.
Larrick, et al., Methods: A Companion to Methods in Enzmmology. (1991).
Lawrence, H. Blood 1997; 89:1922.
Leclerc, S. et al., JBC, 2001; 276:251-260.
Lefebvre V. Matrix Biol 1998; 16:529-40.
Leost, et al., Eur. J. Biochem., (2000); 267:5983-5994.
Leung A Y H et al., Dev Biol. 2004.
Leveen, P., et al. Blood 2002; 100:560-8.
Lewis I D et al., Blood. 2001; 97:3441-3449.
Lim, J. W. and Bodnar, A., Proteomics. 2002; 2(9): 1187-1203 (2002).
Liu H J, Lamming C, C.M. V. Characterization of Human Bone Marrow and Umbilical Cord Blood Self-Renewing Multi-lineage Hematopoietic Stem Cells. 2003.
Liu J M: Fanconi's anemia, in Young N S (ed): Bone marrow failure syndromes. Philadelphia, W.B. Saunders company, 2000, p 47-68.
Loeffler, J. and Behr, J., Methods in Enzymology. 1993; 217:599-618.
Lumelsky, N., et al. Science 2001; 292:1389-94.
Marks et al., J. Mol. Biol. 1991; 222:581-597.
Martin, F., et al., J. Virol. 1999; 73:6923-6929.
Martinez, et al., J. Med. Chem., (2002); 45:1292-1299.
Massague, J. Nat Rev Mol Cell Biol. 2000; 1:169-78.
Matthew, H. W., et al., ASAIO Trans. 1991; 37(3):M328-30.
Matsuda, T., et al. EMBO 1999; 18:4261-9.
Mao et al. Nature 2001; 411:321-25.
McNeish, J. Nature Reviews 2004; 3:70-80.
Medvinsky A et al., Cell. 1996; 86:897.
Meijer, L., et al. Chemistry and Biology 2003; 10:1255-66.
Meijer, et al., Chem. Biol., (2000); 7:51-63.
Meijer, L. et al. Trends in Pharmacological Sciences 2004; 25:471-480.
Mettey, et al., J. Med. Chem., (2003); 46:222-236.
Mikkola H K et al., Blood. 2003; 101:508-516.
Miller, A. D., and C. Buttimore, Mol. Cell. Biol. 1986; 6:2895-2902.
Mitsui, K., et al. Cell 2003; 113:631-42.
Mochizuki, H., et al., J. Virol. 1998; 72:8873-8883.
Molin, M., et al. J. Virol. 1998; 72:8358-8361.
Morrison et al. Proc. Natl. Acad. Sci. 1984; 81, 6851-6855.
Moustakas, A., et al. J Cell Science 2001; 114:4359-69.
Muguruma Y et al., Exp Hematol. 2003; 31:1323-1330.
Muschler, G. F., et al. J Bone Joint Surg. Am. 1997; 79(11):1699-709.
Naerum, et al., Bioorg. Med. Chem. Lett., (2002); 12:1525-1528.
Nakano T et al., Science. 1994; 265:1098-1101.
Nakatsuji, N. and H. Suemori. Scientific World Journal 2002; 26:1762-73.
Nichols J et al., Cell. 1998; 95:379-391.
Niwa, H., et al. Nature Genetics 2000; 24:372-6.
Nordoff, V., et al. Genome 2001; 12:309-17.
Novotny and Haber, Proc. Natl. Acad. Sci. USA. 1985; 82; 4592-4596.
Offield, M. F., Development 1996; 122:983-95.
Olesen, et al., J. Med. Chem., (2003); 46:3333-3341.
Oostendorp R A et al., Blood. 2002; 99:1183-1189.
Oostendorp R A et al., J Cell Sci. 2002; 115:2099-2108.
Ortega, et al., Bioorg. Med. Chem.,(2002); 10:2177-2184.
Pack, et al., Bio/Technology. 1993; 11:1271-77.
Packer, A. I., Dev Dyn 2000; 17:62-74.
Pan, G. J., et al. Cell Research 2002; 12:321-9.
Pan, G. J., et al. Cell Research 2003; 13:499-502.
Peat, et al., Bioorg. Med. Chem. Lett., (2004); 14:2127-2130.
Persons, D., et al., Nature Medicine. 1998; 4: 1201-1205.
Petersen, Science. 1999; 284:1168-1170.
Phiel, et al., Annu. Rev. Pharmacol. Toxicol., (2001); 41:789-813.
Pinson, K. et al. Nature 2000; 407:535-8.
Pittenger, Science 1999; 284: 143-147.
Polakis, P. Genes Dev. 2000; 14:1837-51.
Polychronopoulos, et al., J. Med. Chem., (2004); 47:935-946.
Potocnik A J et al., Proc Natl Acad Sci USA. 1997; 94:10295-10300.
Presta, Curr. Op. Struct. Biol. 1992; 2:593-596.
Punzel M et al., Blood. 1999; 93:3750-3756.
Rackoff W R et al., Blood. 1996; 88:1588-93.
Rebel V I et al., Blood. 1996; 87:3500-3507.
Reichmann et al., Nature. 1988; 332:323-329.
Reya T et al., Nature. 2003; 423:409-414.
Reyes M et al., Ann N Y Acad Sci. 2001; 938:231-233; discussion 233-235.
Reyes M et al., Blood. 2001a; 98:2615-2625.
Reyes M et al., J Clin Invest. 2002; 109:337-346.
Reyes M, and Verfaillie C M. Ann N Y Acad Sci. 2001; 938:231-233; discussion 233-235.
Reyes M, et al., J Clin Invest. 2002; 109:337-346.
Rideout W Mr et al. Cell. 2002; 109:17-27.
Ring, et al., Diabetes, (2003); 52:588-595.
Robbins, et al. J. Virol. 1997; 71(12):9466-9474.
Rosfjord E, Rizzino A. Biochem Biophys Res Commun. 1997; 203:1795-802.
Rowley J. Cancer. 1990; 65:2178-2184.
Ruetz Ruetz, et al., Curr. Med. Chem. Anti-Canc. Agents, (2003); 3:1-14.
Salmons, B. and Gunzburg, W. H., 1993; 4:129-141.
Sato, N., et al. Nature Medicine 2004; 10: 55-63.
Sato and Brivanlou, Methods in Molecular Biology (2006); 331:115-128.
Scagni P et al., Haematologica 1988; 83:432-7.
Schlemminger, I., et al. Bioorg and Med Chem Lett 2003; 13:1451-1454.
Schuh A C, et al., Proc Natl Acad Sci USA. 1999; 96:2159-2164.
Schwartz R E et al., J Clin Investigation. 2002; 96:1291-1302.
Schwartz R E, et al. J Clin Invest. 2002; 109:1291-1302.
Schwarzenberger, P., et al., J. Virol. 1997; 71:8563-8571.
Sebestyen, et al. Nature Biotech. 1998; 16:80-85.
Shake, J. G., et al. Ann. Thorac. Surg. 2002; 73:1919-26.
Shen, et al., Bioorg. Med. Chem., (2004); 12:1239-1255.
Shimozaki et al. Development. 2003; 130:2505-12.
Smith, et al., Bioorg. Med. Chem. Lett., (2001); 11:635-639.
Spangrude G et al., Science. 1988; 241:58.
“Stem Cells and the Future of Regenerative Medicine.” (2002) Committee on the Biological and Biomedical Applications of Stem Cell Research. National Academy Press. Washington, D.C.
Stewart, Colin. Nature Genetics 2000; 24:328-30
Sutton, R., et al., J. Virol. 1998; 72:5781-5788.
Takahashi, J Clin Invest. 2000; 105:71-7.
Takahashi, Nat. Med. 1999; 5:434-8.
Tang et al. BBRC. 2002; 293:167-73.
Testard et al. Bioorg. Med. Chem. Lett. 2006; 16(13)3419-23.
Theise, Hepatology. 2000a; 31:235-40.
Theise, Hepatology. 2000b; 32:11-6.
Theunissen K and Verfaillie C M. Exp Hematol. 2004.
Thomas E et al., Ann Int Med. 1986; 104:155-163.
Thomas E D. Semin Hematol. 1999; 36:95-103.
Thomson J A et al., Science. 1998; 282:1145-1147.
Tian X et al., Exp Hematol. 2004; 32:1000-1009.
Till, J. E. and E. A. McCullough. Radiation Research 1961; 14:213-22.
Tolar, J., et al. 2003; American Society of Hematology 45th Annual Meeting and Exposition, Poster 3115.
Tolar J et al., Blood. 2003; ASH Abstract.
Tolar J et al., Blood. 2004; ASH Abstract.
Toma, C., et al. Circulation 2002; 105:93-8.
Traggiai E et al., Science. 2004; 304:104-107.
Trowbridge et al., Nature Medicine 2006; 12:89-98.
U.S. Pat. No. 6,566,341 (Wang et al.)
U.S. Pat. No. 6,664,285 (Eisenbrand et al.)
Uwanogho D. et al., Mech Dev 1995; 49:23-36.
Van Damme, A., et al. Curr Gene Ther. 2002; 2:195-209.
Vaswani, et al., Annals Allergy, Asthma & Immunol. 1998; 81:105-115.
Verfaillie C et al., Blood. 1992; 79:1003-1010.
Verfaillie C et al., Blood. 1998; 92:1820-1831.
Verfaillie C et al., J Exp Med. 1991; 174:693-703.
Verfaillie C. Blood. 1992; 79:2821-2826.
Verfaillie C M et al., J Clin Invest. 1992; 90:1232.
Verfaillie C M, et al., Blood. 1996; 87:4770-4779.
Verfaillie, C. M. Nat. Immunology 2002; 3:314-7.1
Verfaillie, C. M. Trends Cell Biol. 2002; 12(11):502-8.
Vodyanik M A et al., Blood. 2005; 105:617-626.
Wagers A J, et al. Science. 2002; 297:2256-2259.
Wagman, et al., Curr. Pharmacol. Des., (2004); 10:1105-1137.
Wagner, E., et al., Proc. Natl. Acad. Sci. USA. 1992; 89:6099-6103.
Wang L et al., Immunity. 2004; 21:31-41.
Wang X et al., Nature. 2003; 422:897-901.
Warnecke, C., et al., FASEB J 2003; 17:1186-1188.
Wei et al. Nature Biotechnology. 2004; 22:707-16.
Whitlow, et al., Methods: A Companion to Methods in Enzymology (1991).
Williams, R., et al. Nature 1988; 336:684-7.
Williams, R. S., et al., Proc. Natl. Acad. Sci. USA. 1991; 88:2726-2730.
Willert, K. and R Nusse. Curr. Opin. Gen. Dev. 1998; 8:95-102.
Witherington, et al., Bioorg Med. Chem. Lett., (2003); 13:1577-1580.
Witherington, et al., Bioorg. Med. Chem. Lett., (2003); 13:1581-1584.
Witherington, et al., Bioorg. Med. Chem. Lett., (2003); 13:3055-3057.
Witherington, et al., Bioorg. Med. Chem. Lett., (2003); 13:3059-3062.
Wodarz, A. and R Nusse. Annual Review of Cell and Developmental Biology 1998; 14:59-88.
Wold, W., Adenovirus Methods and Protocols, Humana Methods in Molecular Medicine (1998), Blackwell Science, Ltd.
Wright, L., et al. J. Neurochem. 2003; 86:179-95.
Wu G D, et al. Transplantation. 2003; 75:679-685.
Wu, X., et al. JACS2002; 124:14520-21.
Wu, X., et al. JACS 2004; 126:1590-91.
Wysocki and Sato, Proc. Natl. Acad. Sci. (USA). 1978; 75:2844.
Xiong, C., et al., Science. 1989; 243:1188-1191.
Yahata T et al., J Immunol. 2002; 69:204-209.
Yanagi, K., et al., ASAIO Trans. 1989; 35(3):570-2.
Yang, N. S., et al., Proc. Natl. Acad. Sci. USA. 1990; 87:9568-9572.
Yeom, Y., et al. Development 1996; 122:881-94.
Ying, Q L. et al. Cell 2003; 115: 281-92.
Yoder M et al., Proc Natl Acad Sci USA. 1997; 94:6776.
Zandstra, P. W., et al. Biotechnol Bioeng. 2000; 69:607-17.
Zawel, L., et al. Molecular Cell 1998; 1:611-617.
Zeng L et al., Blood. 2004; ASH abstract.
Zhang, G. et al., Biochem. Biophys. Res. Commun. 1996; 227(3):707-711.
Zhang, H. et al., Med. Chem. Lett., (2003); 13:3049-3053.
Zhang, J. H., et al. J Biomol Screen. 1999; 4:67-73.
Zhao L R, et al. Exp Neurol. 2002; 174:11-20.
Zhao R et al., Blood. 1997; 90:4687-4698.
Zhao R C H et al., Blood. 2001; 97:2406-2412.
Zhu, A. and F. Watt. Development. 1999; 126:2285-98.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A culture method comprising culturing non-embryonic cells at a density of at least about 8,000 cells/cm² in the presence of at least one GSK-3 inhibitor, wherein said cells can differentiate into cell types of more than one embryonic lineage.

2-36. (canceled)
